(19) United States
(12) Patent Application Publication
(10) Pub. No.: US 2026/0183453 A1
(43) Pub. Date: Jul. 2, 2026

(54) METHOD FOR PRODUCING A HYDROGEL COMPRISING A CROSS-LINKED SILYLATED POLYSACCHARIDE

(71) Applicant: TEOXANE SA, GENEVE (CH)

(72) Inventors: Jimmy FAIVRE, VALSERHONE (FR); François BOURDON, GAILLARD (FR)

(21) Appl. No.: 18/857,049

(22) PCT Filed: Apr. 14, 2023
(86) PCT No.: PCT/EP2023/059837

§ 371 Date: Oct. 15, 2024

(30) Foreign Application Priority Data
Apr. 15, 2022 (FR) 2203536

(51) Int. Cl. A61L 27/52; A61L 27/20; A61L 27/54

(52) U.S. Cl. CPC A61L 27/52; A61L 27/20; A61L 27/54; A61L 2300/204; A61L 2300/402

(57) ABSTRACT

The present disclosure relates to a process for preparing a hydrogel comprising a crosslinked polysaccharide, in particular, a process for preparing an injectable hydrogel comprising crosslinked hyaluronic acid. The present invention also relates to a hydrogel, preferably an injectable hydrogel, obtainable by the process, a composition comprising the hydrogel, and uses of the hydrogel.

### TECHNICAL FIELD

The present invention relates to a method for producing a hydrogel comprising a crosslinked polysaccharide, in particular, a method for producing an injectable hydrogel comprising crosslinked hyaluronic acid. The present invention also relates to a hydrogel, preferably injectable, that can be obtained by the method, a composition containing the hydrogel and the uses of this hydrogel.

### PRIOR ART

Polysaccharide hydrogels are used in various fields such as in the aesthetic, cosmetic and therapeutic fields. In particular they can substitute for biological tissues. In particular, gels of hyaluronic acid (HA) have applications in ophthalmology, parodontology, rheumatology or in aesthetic surgery. Hyaluronic acid hydrogels are used, in particular, for filling the soft tissues, preferably the skin, having volume defects such as wrinkles or scars, or for increasing the volume of soft tissues.

In order to obtain hyaluronic acid gels with the mechanical properties, in vivo sustainability and resistance to degradation suitable for filling soft tissue, hyaluronic acid is generally crosslinked with one or more crosslinking agents. Conventional crosslinking agents have at least two reactive functions with functional groups present on the polysaccharide which allow them to bond together polysaccharide molecules and therefore to crosslink them. For this reason, these crosslinking agents have a certain toxicity in vivo because their at least two reactive functions with functional groups present on the polysaccharide are also reactive with groups present on biopolymers and can also allow them to react with biopolymers such as peptides, carbohydrates and DNA, and thus to crosslink them.

For reasons of product biocompatibility and safety, it is therefore desirable to reduce the quantities of crosslinking agent conventionally used in order to keep a polysaccharide as unmodified as possible. However, below a certain threshold, the gels prepared no longer have suitable properties.

In particular, hyaluronic acid gels crosslinked with 1,4-butanediol diglycidyl ether (BDDE) with a degree of modification of approximately 1% are very poorly cohesive.

In order to respond to this problem, various modifications of the method parameters have already been tried.

In particular, it has already been proposed to modify the crosslinking medium by adding different alkali halide salts or phosphates, or by increasing the concentration of hyaluronic acid and/or NaOH (WO2014/064633, WO2016/096920, WO2017/016917).

Adjustment of the duration and temperature of the crosslinking reaction has also been studied (Facile strategy involving low-temperature chemical cross-linking to enhance the physical and biological properties of hyaluronic acid hydrogel, Carbohydrate Polymers, 2018, Sukwha Kim) and some have succeeded in preparing hyaluronic acid gels, or chitosan, crosslinked with lower quantities of conventional crosslinking agent by freezing their reaction media (Preparation and physical properties of hyaluronic acid-based cryogels, Journal of Applied Polymer Science, 2015, Anna Ström et al.; Chitosan gels and cryogels cross-linked with diglycidyl ethers of ethylene glycol and polyethylene glycol in acidic media, Biomacromolecules, 2019, Svetlana Bratskaya et al. and Hyaluronic acid cryogels with non-cytotoxic crosslinker genipin, Materials Letters, Joahanna Roether). However, the gels thus obtained are not homogeneous.

It is therefore still desirable to find a means of further reducing the quantities of conventional crosslinking agent used in order to obtain crosslinked polysaccharide gels, such as crosslinked hyaluronic acid gels, with mechanical properties suitable for filling soft tissues.

Furthermore, it has been considered to functionalise biopolymers, such as hyaluronic acid, with alkoxysilanes groups capable of reacting with each another by sol-gel condensation reaction in order to form Si—O—Si bonds. The gels prepared in this way are thus crosslinked without a conventional crosslinking agent. This is illustrated, in particular, in WO2011/089267 and WO2017/009200. However, gels of this type using only silicon derivatives are unstable during heat sterilisation and become solutions after treatment. Furthermore, neither WO2011/089267 nor WO2017/009200 applies such a final sterilisation step. Moreover, it should be noted that WO2011/089267 does not result in the formation of a gel with the mechanical properties desirable for being injected. Condensation sol-gel reactions are known to be promoted at pH close to neutrality and/or in dehydrated medium, in particular obtained by drying (Lee et al., One-pot synthesis of silane-modified hyaluronic acid hydrogels for effective antibacterial drug delivery via sol-gel stabilization, Colloids and surfaces B: Biotinterfaces, 2019, 174:308-315). However, these conditions have several disadvantages. More specifically, at pH close to neutrality and at ambient temperature, without drying, the probability of an encounter between two silanol groups remains low and an insufficient number of Si—O—Si bonds are formed. During drying: heat-sensitive polymer chains, such as hyaluronic acid, degrade and generate low molecular weight polymer fragments, the biocompatibility of which is uncertain; the concentration of polymer in the reaction medium is difficult to control; temperature gradients are created in the reaction medium which is then inhomogeneous; generation of bubbles is possible; and drying out of the final gel is possible.

There is therefore still a need to provide new crosslinked polysaccharide-based hydrogels, in particular crosslinked hyaluronic acid-based hydrogels, containing smaller quantities of conventional crosslinking agent such as BDDE, or even containing no conventional crosslinking agent, and advantageously being able to be injected and/or sterilised.

### SUMMARY

The present invention relates to a method for preparing a hydrogel, preferably injectable, comprising the following steps:
a) providing at least one polysaccharide;
b) providing at least one molecule of formula Chem. I:

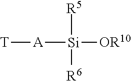

or a salt thereof,
wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy or carbodiimide group, or an acid anhydride residue;
A represents a chemical bond or a spacer group;
R⁵ and R⁶, identical or different, represent a hydrogen atom; a halogen atom; an —OR⁴ group with R⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl;
R¹⁰ represents a hydrogen atom, an aryl group or an aliphatic hydrocarbon group having 1 to 6 carbon atoms;
c) functionalisation of the polysaccharide with at least one molecule of formula Chem. I;
d) crosslinking by sol-gel reaction of the functionalised polysaccharide in order to give a hydrogel;
wherein step d) comprises a crosslinking by sol-gel reaction carried out in a reaction medium at a pH greater than or equal to 9 and less than 14, at a pressure P less than or equal to atmospheric pressure and at a temperature T greater than the temperature of the eutectic point of the reaction medium as measured at pressure P, and less than the temperature of the freezing point of the reaction medium as measured at pressure P, for a duration t ranging from 2 weeks to 17 weeks,
or
wherein step d) comprises a crosslinking by sol-gel reaction carried out in a reaction medium at a pH greater than or equal to 6.8 and less than 7.8, at a pressure P less than or equal to atmospheric pressure and at a temperature T greater than the temperature of the eutectic point of the reaction medium as measured at pressure P and less than the temperature of the freezing point of the reaction medium as measured at pressure P, for a duration t between 1 hour and 48 hours.

The present invention also relates to a hydrogel that can be obtained by a method as described previously and below, as well as to a cosmetic or pharmaceutical composition comprising such a hydrogel.

Finally, the present invention relates to such a hydrogel or such a composition for use in the filling and/or replacement of tissues; for preventing and/or treating the negative changes in the viscoelastic or biomechanical properties of the skin; for filling volume defects of the skin, in particular for filling wrinkles, fine lines and scars; for attenuating the nasolabial folds and bitterness folds; for increasing the volume of the cheekbones, the chin or lips; for restoring the volumes of the face, in particular the cheeks, temples, the oval of the face, and around the eye; for reducing the appearance of wrinkles and fine lines; or to stimulate, regenerate, hydrate, firm or restore the radiance of the skin, in particular by mesotherapy.

The invention also relates to the use of such a hydrogel or such a composition, the composition comprising at least one cosmetic active ingredient, for the modified, delayed or prolonged release of cosmetic active ingredients.

Other aspects of the invention are as described in the claims and below.

### Definitions

The term “gel” designates a polymer network which is dilated over its entire volume by a fluid. This means that a gel is formed of two media, one “solid” and the other “liquid”, dispersed in one another. The medium referred to as “solid” consists of long-molecule polymers connected to one another by weak bonds (for example hydrogen bonds) or covalent bonds (crosslinking). The liquid medium consists of a solvent. A gel generally corresponds to a product which has a phase angle δ less than or equal to 45° at 1 Hz for a deformation of 0.1% or a pressure of 1 Pa, advantageously a phase angle δ ranging from 2° to 45° or from 20° to 45°.

The term “hydrogel” designates a gel as defined above, wherein the solvent constituting the liquid medium is mostly water (for example at least 90%, in particular at least 95%, especially at least 99%, by weight of the liquid medium).

Preferably, the liquid medium comprises, in particular consists of, a buffer solution, advantageously enabling a pH of the liquid medium between 6.8 and 7.8, in particular a phosphate-buffered saline.

The term “injectable gel” designates a gel that can flow and be injected manually by means of a syringe equipped with a needle of diameter ranging from 0.1 to 0.5 mm, for example a 30 G, 27 G, 26 G, 25 G hypodermic needle. Preferably, an “injectable gel” is a gel having a mean extrusion force less than or equal to 25 N, preferably ranging from 5 to 25 N, more preferably ranging from 8 to 15 N, during a measurement with a dynamometer, at a fixed speed of approximately 12.5 mm/min, in syringes of external diameter greater than or equal to 6.3 mm, with a needle of external diameter less than or equal to 0.4 mm (27 G) and length ½″, at ambient temperature.

The property “stretchiness” of a product designates its ability to be stretched between two surfaces to which it has adhered. The stretchiness property can be determined using a texturometer, a sensory analysis performed by a panel, or else rheological and mechanical measurements including, in particular, measurement of the phase angle (δ) or tensile tests. In particular, this property can be measured as described by P. Micheels et al. (Micheels et al., Comparison of two swiss-designed hyaluronic acid gels: six-month clinical follow-up, Journal of Drug in Dermatology, 2017, 16:154-161, “Resistance to stretching”) or by carrying out a tack test and by measuring the length of the threads under traction.

The term “polysaccharide” designates a polymer composed of monosaccharides (preferably D-enantiomers) joined together by glycosidic bonds.

The term “monosaccharide”, also referred to as an “ose”, designates a non-modified or modified monosaccharide.

A “non-modified monosaccharide” designates a compound of formula H—(CHOH)ₓ—CO—(CHOH)_{y}—H with x and y representing, independently of one another, an integer ranging from 0 to 5 under the condition that 2≤x+y≤5, the monosaccharide can be in a linear form represented by the above-mentioned formula or can be in a cyclic form by reaction of the CO function (aldehyde or ketone) with one of the OH groups in order to form a hemiacetal or hemiketal. Preferably, the monosaccharide is in cyclic form. There are two types of ose: aldoses, which carry an aldehyde function (when x or y equals 0) and ketoses which carry a ketone function (when neither x, nor y equals 0). Monosaccharides are classified by number of carbons. For example, monosaccharides with 6 carbons (x+y=5) are hexoses of formula C₆H₁₂O₆ and can be allose, altrose, glucose, mannose, gulose, idose, galactose or talose. Monosaccharides with 5 carbons (x+y=4) are pentoses of formula C₅H₁₀O₅ and can be ribose, arabinose, xylose, or lyxose. The monosaccharide is preferably a hexose, in other words x+y=5.

A monosaccharide further comprises x+y asymmetric carbons and therefore 2^{(x+y+1)} enantiomer pairs. Each enantiomer pair is designated by a different name and the enantiomers of a same pair are respectively qualified as D and L enantiomers.

A “modified monosaccharide” designates a non-modified monosaccharide as defined above for which, for example:
one or more OH functional groups have been replaced by another functional group, for example:
(i) an OR group with R representing a (C₁-C₆)alkyl group such as methyl or ethyl; a hydroxy-(C₁-C₆)alkyl groups such as hydroxyethyl (—CH₂CH₂OH) or hydroxypropyl (—CH₂—CH(OH)—CH₃); a carboxy-(C₁-C₆)alkyl group such as carboxymethyl (—CH₂COOH); or
CO—(C₁-C₆)alkyl groups such as acetyl; and/or
(ii) an NR′R″ group with R′ and R″ representing, independently of one another, H, (C₁-C₆)alkyl or CO—(C₁-C₆)alkyl such as acetyl; and/or
(iii) an OSO₃H group; and/or
   the one or more CH₂OH end functions have been replaced by a COOH or CHO group;
   a —CH(OH)—CH(OH)— bond is oxidised to give two —CHO (aldehyde) end groups in place of this bond; and/or
   a CH₂OH end function has been condensed with an OH functional group in order to form an —O—CH₂— chain.

The expression “repetition unit” of a polysaccharide designates a structural unit consisting of one or more (generally 1 or 2) monosaccharides, the repetition of which produces the complete polysaccharide chain.

Some or all of the monosaccharides can be in a modified form.

The monosaccharides, when they are modified, can be in different modified forms.

The term “physiologically acceptable” designates that which is generally safe, non-toxic and neither biologically nor otherwise undesirable and which is acceptable for cosmetic use (in other words non-therapeutic use) or for human or veterinary therapeutic use, in particular for use by injection into the human or animal body or for a topical application on the skin.

The “salts” used in the context of the present invention are preferably physiologically acceptable salts. The term “physiologically acceptable salts” designates, in particular:
1) the pharmacologically acceptable acid addition salts formed with pharmaceutically acceptable inorganic acids such as hydrochloric acid, hydrobromic acid, the sulfuric acid, nitric acid, phosphoric acid and similar; or formed with pharmaceutically acceptable organic acids, such as formic acid, acetic acid, benzenesulfonic acid, benzoic acid, camphorsulfonic acid, citric acid, ethane-sulfonic acid, the fumaric acid, glucoheptonic acid, gluconic acid, glutamic acid, glycolic acid, hydroxynaphtoic acid, 2-hydroxyethanesulfonic acid, lactic acid, maleic acid, malic acid, mandelic acid, methanesulfonic acid, muconic acid, 2-naphtalenesulfonic acid, propionic acid, salicylic acid, succinic acid, dibenzoyl-L-tartric acid, tartric acid p-toluenesulfonic acid trimethylacetic acid, trifluoroacetic acid and similar, and
2) pharmacologically acceptable base addition salts formed when an acid proton present in the parent compound is either replaced by a metal ion, for example an alkali metal ion (e.g. Na, K), alkaline earth metal ion (e.g., Ca, Mg), a zinc ion, a silver ion or an aluminium ion; or coordinated with a pharmaceutically acceptable organic base such as diethanolamine, ethanolamine, N-methylglucamine, triethanolamine, tromethamine and similar; or with a pharmaceutically acceptable inorganic base, such as aluminium hydroxide, calcium hydroxide, potassium hydroxide, sodium carbonate, sodium hydroxide and similar.

The “degree of modification” (MOD) of a polysaccharide, such as hyaluronic acid, corresponds to the molar quantity of modifying agent, such as the quantity of crosslinking agent and/or the functionalisation agent bonded to the polysaccharide, by one or more of its ends, expressed per 100 moles of polysaccharide repetition units. It can be determined by the methods known to a person skilled in the art, such as nuclear magnetic resonance spectroscopy (NMR).

The “degree of functionalisation” (DOF) corresponds to the molar quantity of functionalisation agent bonded to the polysaccharide, by one end, expressed for 100 moles of polysaccharide repetition units. It can be determined by the methods known to a person skilled in the art, such as nuclear magnetic resonance spectroscopy (NMR).

The “molar crosslinking ratio” (TR), expressed in %, designates the molar ratio of the quantity of crosslinking agent to the quantity of polysaccharide repetition unit introduced into the crosslinking reaction medium, expressed per 100 moles of polysaccharide repetition units in the crosslinking medium.

The “molar functionalisation ratio”, expressed in %, designates the molar ratio of the quantity of functionalisation agent to the quantity of polysaccharide repetition unit used in the crosslinking reaction medium, expressed per 100 moles of polysaccharide repetition units in the functionalisation medium.

The expression “molar modification ratio” designates the molar ratio of the quantity of modifying agent (e.g., crosslinking agent and/or molecule of formula Chem. I as functionalisation agent) relative to the quantity of polysaccharide repetition unit used in the modification medium.

The expression “therapeutic active ingredient” designates a substance for curing, relieving the symptoms of and/or preventing a disease; a substance having curative or preventative properties with respect to human or animal diseases, as well as any substance which can be used in humans or in animals or which can be administered to them, with a view to establishing a medical diagnosis or restoring, correcting or modifying their physiological functions by exerting a pharmacological, immunological or metabolic action.

The expression “cosmetic active ingredient” designates any non-therapeutic substance, in particular intended to be placed in contact with various superficial parts of the human body, such as the epidermis, the hair and capillary systems, nails, lips, chest and teeth, with a view, exclusively or mainly, to cleaning, protecting, or perfuming them, maintaining them in good condition, modifying their appearance or smell.

The term “approximately” designates that the value concerned can be less than or greater than the indicated value by 10%, notably by 5%, in particular by 1%.

An “aqueous reaction medium” designates a reaction medium for which the solvent is mostly water (for example at least 90%, in particular at least 95%, in particular at least 99% by total weight of the solvent) or is water.

The expression “spacer group” designates a fragment comprising at least one atom intended to link together two chemical groups within a same molecule. The spacer group preferably contains at least one carbon atom.

The term “halogen” designates an atom of fluorine, chlorine, bromine or iodine.

An “epoxide” group is an ethylene oxide residue linked to the remainder of the molecule by one of its carbon atoms.

An “N-succinimidyloxycarbonyl” group is a group of formula Chem. GR1 below:

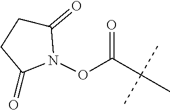

An “N-sulfosuccinimidyloxycarbonyl” group is a group of formula Chem. GR2 below:

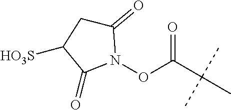

A “halogenocarbonyl” group is a group of formula —CO-Hal with Hal representing a halogen, such as Cl or Br.

A “carbodiimide” group is a group comprising an —N═C═N— unit, and more particularly a group of formula —N═C═N—R^{a} with R¹ representing an aliphatic hydrocarbon group having 1 to 20 carbon atoms, preferably a (C1-C6)alkyl group, for which one or more carbon atoms are optionally replaced by a heteroatom chosen from O, S and N, in particular N. An “acid anhydride residue” is a group comprising a —C(O)—O—C(O)— unit, and more particularly a monovalent cyclic group comprising the —C(O)—O—C(O)— unit, such as a saturated monovalent hydrocarbon monocyclic group comprising 5 to 10, in particular 5 or 6 carbon atoms, of which three successive carbon atoms are replaced by C(O)—O—C(O) and optionally one or more of which, in particular one, additional carbon atoms, preferably not consecutive with the three carbon atoms substituted by —C(O)—O—C(O)—, are each replaced by a heteroatom such as N, O or S, in particular N. The acid anhydride residue may respond in particular to the following formula Chem. GR3:

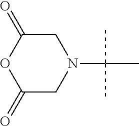

The acid anhydride residue can also be chosen from a maleic anhydride residue or a succinic anhydride residue.

The expression “aliphatic hydrocarbon chain” or “aliphatic hydrocarbon group” designates a linear, branched and/or cyclic, saturated or unsaturated, but not aromatic, hydrocarbon group, advantageously comprising 1 to 50, in particular 1 to 20, for example 1 to 12 or 1 to 6 carbon atoms. It involves, in particular, alkyl groups

The expression “branched aliphatic hydrocarbon chain” specifically designates a main aliphatic hydrocarbon chain comprising at least one secondary aliphatic hydrocarbon chain.

The expression “star-shaped aliphatic hydrocarbon chain” designates a branched aliphatic hydrocarbon chain comprising a plurality of secondary aliphatic hydrocarbon chains all starting from a single branching point.

The expression “alkyl with C1-Cx” or “(C1-Cx)alkyl” or else “alkyl having 1 to x carbon atoms” designates a saturated, linear or branched, monovalent hydrocarbon group, having 1 to x carbon atoms, with x an integer, for example a methyl, ethyl, isopropyl, tertio-butyl, n-pentyl, cyclopropyl or cyclohexyl group, etc.

The expression “(C1-Cx)alkylene” designates a saturated, linear or branched, divalent hydrocarbon group comprising 1 to x carbon atoms, with x an integer, for example a methane-1,1-diyl, ethane-1,1-diyl, ethane-1,2-diyl, propane-1,3-diyl, butane-1,4-diyl, butane-1,3-diyl, butane-1,2-diyl, pentane-1,5-diyl, hexane-1,6-diyl, hexane-1,5-diyl, heptane-1,7-diyl, octane-1,8-diyl, nonane-1,9-diyl or decane-1,10-diyl group, etc. It involves, in particular, a methane-1,1-diyl or propane-1,3-diyl group.

The expression “hydroxy-(C1-Cx)alkyl” designates a (C1-Cx)alkyl group such as defined above substituted by a hydroxyl (OH) group, for example a hydroxyethyl (—CH₂CH₂OH) or a hydroxypropyl (for example —CH₂—CH(OH)—CH₃).

The expression “carboxy-(C1-Cx)alkyl” designates a (C1-Cx)alkyl group such as defined above, substituted by a carboxyl (COOH) group, for example a carboxymethyl (—CH₂COOH) group.

The expression “aryl” designates a monovalent aromatic hydrocarbon group, preferably having 6 to 10 carbon atoms, comprising one or more cycles, for example a phenyl or naphtyl group.

The expression “arylene” designates a divalent aromatic hydrocarbon group, preferably having 6 to 10 carbon atoms, comprising one or more cycles, such as a phenylene group.

The expression “aryl-(C1-Cx)alkyl” designates an aryl group such as defined above, linked to the rest of the molecule by means of a (C1-Cx)alkyl chain as defined above, with x an integer, for example the benzyl or phenylethyl group.

The expression “polyvalent group” designates a group which can form a plurality of covalent bonds with other groups of a same compound or of two different compounds. The bonds to the other groups can be formed from the same atom of the polyvalent group or from different atoms of the polyvalent group, and preferably from different atoms of the polyvalent group. In particular, the polyvalent group is a divalent group and can therefore form two covalent bonds with two other groups of the same compound or of two different compounds. The number of covalent bonds that can be formed designates the “valence” of the polyvalent group.

The expression “partially concomitant” as used in expressions of the type “steps b) and c) are partially concomitant” means that the two steps are carried out, in part, at the same time, under the same reaction conditions, but that at least one of the two steps is initiated or terminated under different reaction conditions from the common reaction conditions.

### DETAILED DESCRIPTION OF THE INVENTION

To overcome the disadvantages described above, the inventors propose functionalising the polysaccharide using a molecule (here designated as functionalisation agent or molecule of formula Chem. I) comprising a single function capable of reacting with a functional group of the polysaccharide and a silylated group capable of reacting with another silylated group via a sol-gel reaction so as to enable the crosslinking of the polysaccharide and to form a hydrogel. The crosslinking by sol-gel reaction is, at least partially, carried out a pressure P less than or equal to atmospheric pressure and at a temperature T greater than the temperature of the eutectic point of the reaction medium as measured at pressure P, and less than the temperature of the freezing point of the reaction medium as measured at pressure P. The duration t of the crosslinking by sol-gel reaction at temperature T and pressure P is a function of the pH of the reaction medium.

The “sol-gel reaction” consists of forming Si—O—Si bonds from Si—OR groups, with R representing a hydrogen atom, an aryl group or an aliphatic hydrocarbon group having 1 to 6 carbon atoms. This reaction proceeds as follows:
(i) if R is not a hydrogen atom, a hydrolysis step of at least some of the Si—OR groups in order to give Si—OH groups; then
(ii) a step of condensing Si—OH groups two-by-two or a Si—OH group with a Si—OR group in order to form Si—O—Si bonds.

In the present invention, the polysaccharide is functionalised by means of a molecule of formula Chem. I in such a way as to become a carrier of Si—OR groups which will be able to react together and lead to a crosslinked polysaccharide.

Since the molecule of formula Chem. I comprises a single reactive function with regard to the polysaccharide and enables crosslinking only via a sol-gel reaction, it does not have the toxicity of conventional cross-linking agents: the molecule of formula Chem. I cannot directly crosslink with biological molecules (proteins, DNA, etc.).

In addition, the proposed method makes it possible to prepare crosslinked polysaccharide-based hydrogels:
having superior viscoelastic properties when compared with a composition that is identical but prepared at ambient temperature;
with a particular texture visible to the naked eye and detectable to the touch, namely the gels according to the invention are stretchy and cohesive;
with properties that can be adapted, on demand, by simple passage to a temperature greater than the temperature of the eutectic point of the reaction medium and less than or equal to the temperature of the freezing point of the reaction medium measured at atmospheric pressure for longer or shorter durations.

This adaptability is particularly advantageous in the context of preparing compositions for modified, delayed or prolonged release of active substances, the active substance being pre-incorporated in the composition or added extemporaneously: the longer the composition is frozen the greater will be the sol-gel reaction and the slower will be the release of the active substance. Thus, it is possible to adapt the duration and/or the intensity of release of the product to the requirement:
maintaining desirable mechanical properties after sterilisation;
stable over time compared to the compositions of the prior art;
with preserved polysaccharide chains therefore comprising fewer low molecular weight polysaccharide fragments, including when a very low pH is applied during the preparation. The analysis of low molecular weight polysaccharides in the gels according to the invention, for example by SEC-MALLS, can be used to characterise this phenomenon;
with additional biological effects, such as the improvement of skin quality in animals, in particular in humans;
in a single step, the functionalisation and sol-gel reaction being able to be simultaneous;
possibly without using conventional crosslinking agents.

An object of the present invention is therefore a method for preparing a hydrogel, preferably injectable, comprising the following steps:
a) providing at least one polysaccharide;
b) providing at least one molecule of formula Chem. I:

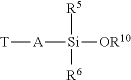

or a salt thereof,
wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy or carbodiimide group, or an acid anhydride residue;
A represents a chemical bond or a spacer group;
R⁵ and R⁶, identical or different, represent a hydrogen atom; a halogen atom; an —OR⁴ group with R⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl;
R¹⁰ represents a hydrogen atom, an aryl group or an aliphatic hydrocarbon group having 1 to 6 carbon atoms;
c) functionalisation of the polysaccharide with at least one molecule of formula Chem. I;
d) crosslinking by sol-gel reaction of the functionalised polysaccharide in order to give a hydrogel;
wherein step d) comprises a crosslinking by sol-gel reaction carried out in a reaction medium at a pH greater than or equal to 9 and less than 14, at a pressure P less than or equal to atmospheric pressure and at a temperature T greater than the temperature of the eutectic point of the reaction medium as measured at pressure P and less than the temperature of the freezing point of the reaction medium as measured at pressure P, for a duration t ranging from 2 weeks to 17 weeks,
or
wherein step d) comprises a crosslinking by sol-gel reaction carried out in a reaction medium at a pH greater than or equal to 6.8 and less than 7.8, at a pressure P less than or equal to atmospheric pressure and at a temperature T greater than the temperature of the eutectic point of the reaction medium as measured at pressure P and less than the temperature of the freezing point of the reaction medium as measured at pressure P, for a duration t between 1 hour and 48 hours.

It should be understood that that which precedes step d) is carried out at least partially at pressure P and temperature T. The duration t of the crosslinking by sol-gel reaction at pressure P and temperature T depends on the pH of the reaction medium.

In the method above, the one or more polysaccharides can be in the form of a salt.

Another object of the present invention is a hydrogel which may be obtained by the method according to the invention.

Another object of the present invention is a composition comprising a hydrogel according to the invention, as well as the therapeutic, cosmetic or aesthetic applications of the hydrogels or compositions according to the invention.

### METHOD

The steps of the method of the present invention can be as described below.

### Step a)

Step a) of the method according to the invention consists of providing at least one polysaccharide. The polysaccharide can be in the form of a salt.

The polysaccharide can be any polymer composed of monosaccharides joined together by glycosidic bonds.

Preferably, the polysaccharide is chosen from pectin and pectic substances; chitosan; chitin; cellulose and its derivatives; agarose; glycosaminoglycans such as hyaluronic acid, heparosan, dermatan sulfate, keratan sulfate, chondroitin and chondroitin sulfate; and the mixtures thereof.

The “pectic substances”, including “pectin”, are polysaccharides composed by a D-galacturonic acid skeleton in acid form, possibly esterified by methanol, and L-rhamnose capable of forming branches with other oses.

“Chitosan” and “chitin” are both polysaccharides composed of D-glucosamine repetition units bonded together in β-(1,4) a part of which is N-acetylated. More particularly, chitosan as a degree of acetylation less than 50%, whereas chitin has more particularly a degree of acetylation greater than 50%.

“Cellulose” is a polysaccharide composed of a linear chain of D-glucose molecules.

The “cellulose derivatives” comprise methylcellulose, ethylcellulose, ethylmethylcellulose, hydroxypropylmethylcellulose (HPMC), hydroxyethylcellulose (HEC), hydroxypropylcellulose (HPC) and carboxymethylcellulose (CMC).

“Agarose” is a polysaccharide comprising, as repetition unit, a disaccharide of D-galactose and of 3,6-anhydro-L-galactopyranose.

“Glycosaminoglycans” are linear polysaccharides composed of repetition units of disaccharides, said disaccharides containing a hexosamine (glucosamine (GlcN) or galactosamine (GalN)) and another ose (glucuronic acid (GlcA), iduronic acid (IdoA) or galactose (Gal)). The hexosamine and the other ose can optionally be sulfated and/or acetylated. The glycosaminoglycan can be, in particular, hyaluronic acid, heparosan, dermatan sulfate, keratan sulfate, chondroitin or chondroitin sulfate.

“Hyaluronic acid” is a glycosaminoglycan for which the repetition unit is a disaccharide composed of D-glucuronic acid and N-acetyl-D-glucosamine, bonded together by alternating glycosidic bonds, β-(1,4) and β-(1,3). When the hyaluronic acid is in the form of a salt, reference is also made to a “hyaluronate” or “hyaluronan”. In the context of the present invention, the hyaluronic acid can have a weight average molar mass ranging from 0.5 to 10 MDa, preferably ranging from 0.5 to 5 MDa, yet more preferably greater than 0.05 MDa, for example ranging from 0.07 to 10 MDa or from 0.07 to 5 MDa, or from 0.5 to 5 MDa or from 1 to 5 MDa or from 2 to 4 MDa. The hyaluronic acid can be in the form of a salt, in particular in the form of a physiologically acceptable salt, such as the sodium salt, potassium salt, zinc salt, calcium salt, magnesium salt, silver salt, calcium salt, and the mixtures thereof. More particularly, the hyaluronic acid is in acid form or in the form of a sodium salt (NaHA).

“Heparosan” is a glycosaminoglycan for which the repetition unit is a disaccharide composed of glucuronic acid (GlcA) bonded by an α-(1,4) bond to an N-acetyl glucosamine (GlcNAc). Each disaccharide repetition unit is linked to the next by a β-(1,4) bond.

“Chondroitin sulfate” is a glycosaminoglycan for which the repetition unit is a disaccharide composed of glucuronic acid bonded in—β(1,3) to sulfated N-acetyl galactosamine, in other words it comprises at least one sulfate substituent. Each disaccharide repetition unit is linked to the next by a β-(1,4) bond.

“Dermatan sulfate” is a glycosaminoglycan for which the repetition unit is a sulfated disaccharide, in other words comprising at least one sulfate substituent, L-iduronic acid and N-acetyl-galactosamine- bonded by α(1-3) bonds. Advantageously, the disaccharide is sulfated in position C-4 of the N-acetyl-galactosamine, in position C-6 of the N-acetyl-galactosamine, in position C-2 of the L-iduronic acid, or at a combination of these positions. Each disaccharide repetition unit is linked to the next by a β-(1,4) bond.

“Keratan sulfate” is a glycosaminoglycan for which the repetition unit is a sulfated disaccharide, in other words comprising at least one sulfate substituent, composed of D-galactose and N-acetylglucosamine bonded by alternating bonds, β(1-4) and β(1-3). The polysaccharide can be in the form of a salt, in particular in the form of a physiologically acceptable salt such as the sodium salt, potassium salt, zinc salt, calcium salt, magnesium salt, silver salt and the mixtures thereof, more particularly a sodium or potassium salt.

Advantageously, the polysaccharide is a glycosaminoglycan or a salt thereof, preferably hyaluronic acid or a salt thereof, more preferably hyaluronic acid or one of its physiologically acceptable salts, such as the sodium salt, potassium salt, zinc salt, silver salt, and the mixtures thereof, still more preferably the hyaluronic acid or its sodium salt. The polysaccharide generally has a weight average molar mass ranging from 0.03 to 10 MDa.

Preferably, if the polysaccharide is hyaluronic acid or one of the salts thereof, it has a weight average molar mass (Mw) ranging from 0.05 to 10 MDa, preferably ranging from 0.5 to 5 MDa, yet more preferably greater than 0.05 MDa, for example ranging from 0.07 to 10 MDa or from 0.07 to 5 MDa, or from 0.5 to 5 MDa or from 1 to 5 MDa or from 2 to 4 MDa.

The polysaccharide can be provided in totally or partially hydrated form, or in dry form, such as in the form of a powder or fibres.

In certain embodiments, in step a), the polysaccharide is provided in dry form, such as in the form of a powder or fibres.

When the polysaccharide is provided in hydrated form, it is in the form of a non-crosslinked gel or a solution. In particular, when the polysaccharide is in hydrated form, it is a non-crosslinked aqueous gel or an aqueous solution. More particularly, the polysaccharide is mixed with water, optionally with added phosphate buffer or supplemented phosphate buffer, in other words possibly comprising additional components as defined in step f).

### Step b)

Step b) of the method according to the invention consists of providing at least one molecule of formula Chem. I as presented above.

Preferably, in formula Chem. I, T represents an isocyanate, sulfhydryl, amino, epoxide, vinyl, formyl, or carbodiimide group, more advantageously, T represents an epoxide or amino group, yet more advantageously T represents an epoxide group.

Preferably, in formula Chem. I, A represents a spacer group, more preferably a divalent aliphatic hydrocarbon chain, in particular linear or branched, and saturated, having 1 to 12 carbon atoms:
wherein one or more (in particular 1, 2, 3 or 4) divalent units are optionally intercalated between two carbon atoms of said chain, chosen from the arylenes, —O—, —S—, —S(O)—, —C(═O)—, —SO₂— and —N(R⁹)— with R⁹ representing a hydrogen atom, an aliphatic hydrocarbon group having 1 to 6 carbon atoms, or an aryl-(C1-C6)alkyl group,
said chain being non-substituted or substituted by one or more monovalent groups chosen from a halogen atom, a hydroxyl, an aryl-(C1-C6)alkyl group.

Advantageously, A is a aliphatic divalent hydrocarbon chain, in particular linear or branched, and saturated, in which one or more —O— divalent units, more advantageously 1 to 4 —O— divalent units, yet more advantageously one divalent —O— unit, are optionally intercalated between two carbon atoms of said chain.

Preferably, A is a (C1-C12)alkylene chain, wherein one or more divalent —O— units, more preferably 1 to 4 divalent —O— units, yet more preferably one divalent —O— unit, are optionally intercalated between two carbon atoms of said chain

In particular, A represents a divalent —(C1-C6)alkylene-O—(C1-C6)alkylene- chain, in particular —(C1-C4)alkylene-O—(C1-C4)alkylene-, more particularly a divalent —CH₂—O—(CH₂)₃— chain, the CH₂ group being bonded to T and the (CH₂)₃ group being bonded to Si in the molecule of formula Chem. I.

Advantageously, the spacer group will also make it possible to avoid steric hindrance between the silylated group and the T group of the molecule of formula Chem. I, ensuring a stable bond between these two groups.

Preferably, in formula Chem. I, R⁵ and R⁶, identical or different, representing an —OR⁴ group with R⁴ representing a hydrogen atom, an aryl group or an aliphatic hydrocarbon group having 1 to 6 carbon atoms; or an aliphatic hydrocarbon group having 1 to 6 carbon atoms optionally substituted by one or more groups chosen from a halogen atom, an aryl and a hydroxyl group.

In particular, R⁵ and R⁶, identical or different, represent an —OR⁴ group with R⁴ representing an (C1-C6)alkyl group; or a (C1-C6)alkyl group.

Advantageously, R⁵ and R⁶, identical or different, represent an —OR⁴ group with R⁴ representing a hydrogen atom, an aryl group or an aliphatic hydrocarbon group having 1 to 6 carbon atoms, preferably with R⁴ representing an aliphatic hydrocarbon group having 1 to 6 carbon atoms, such as a (C1-C6)alkyl group.

Preferably, in formula Chem. I, R¹⁰ represents a hydrogen atom or an aliphatic hydrocarbon group having 1 to 6 carbon atoms such as a (C1-C6)alkyl group, more advantageously R¹⁰ represents an aliphatic hydrocarbon group having 1 to 6 carbon atoms such as a (C1-C6)alkyl group.

Preferably, the molecule of formula Chem. I is such that:
T is as defined above and advantageously represents an amino or epoxide group, preferably an epoxide group;
A is a divalent —(C1-C6)alkylene-O—((C1-C6)alkylene- chain, in particular —(C1-C4)alkylene-O—(C1-C4)alkylene-, such as —CH₂—O—(CH₂)₃—, the CH₂ group preferably being bonded to T and the (CH₂)₃ group being bonded to Si in the molecule of formula Chem. 1;
R⁵ and R⁶, identical or different, are each an —OR⁴ group with R⁴ representing a (C1-C6)alkyl group, preferably a methyl or an ethyl group; or a (C1-C6)alkyl group, preferably a methyl or a ethyl group; and
R¹⁰ is a (C1-C6)alkyl group, preferably a methyl or ethyl group;
the R⁵, R⁶ and OR¹⁰ groups can be identical.

In particular, the molecule of formula Chem. I is chosen from the (3-aminopropyl)triethoxysilane (APTES), (3-glycidyloxypropyl)trimethoxysilane (GPTMS), 3-glycidoxypropyldimethoxymethylsilane, 3-glycidoxypropyldimethylethoxysilane, (3-glycidyloxypropyl)ethoxydimethoxysilane, (3-glycidyloxypropyl)triethoxysilane, diethoxy(3-glycidyloxypropyl)methylsilane, and the mixtures thereof; preferably from (3-glycidyloxypropyl)trimethoxysilane (GPTMS), (3-glycidyloxypropyl)ethoxydimethoxysilane, (3-glycidyloxypropyl)triethoxysilane, diethoxy(3-glycidyloxypropyl)methylsilane, and the mixtures thereof.

### Step c)

The polysaccharide is functionalised with at least one molecule of formula Chem. I as presented above.

Step c) enables functionalising of the polysaccharide chains. The functional group T of the molecule of formula Chem. I reacts with a functional group present on the polysaccharides so as to functionalise the polysaccharide chains. Notably, the functional group T of the molecule Chem. I thus reacts with an —OH or —COOH group, or even a CHO function, present on the polysaccharides, such as hyaluronic acid. Functionalised polysaccharides are thus obtained, comprising dangling bonds on a polysaccharide chain, said dangling bonds comprising a -A-Si(R⁵)(R⁶)OR¹⁰ group, the -A-Si(R⁵)(R⁶)OR¹⁰ group coming from the molecule of formula Chem. I of step b) being able to give the hydrogel biological properties.

Preferably, in step c), the polysaccharide is functionalised in the presence of 0.01 to 0.50, preferably 0.05 to 0.45, in particular 0.10 to 0.25 moles of the molecule of formula Chem. I or a salt thereof, per 1 mole of polysaccharide repetition unit.

Typically, the higher the weight average molar mass Mw of the polysaccharide, the lower will be the functionalisation ratio of the molecule of formula Chem. I, with a view to obtaining a hydrogel having equivalent mechanical properties, in particular equivalent viscoelastic properties (in particular elastic modulus G′, stress at the intersection of G′ and G″ and/or phase angle δ). In other words, the higher the weight average molar mass Mw of the polysaccharide, the lower will be the molar quantity of the molecule of formula Chem. I introduced in step c).

The functionalisation of the polysaccharide is typically carried out in an aqueous reaction medium.

In certain embodiments, in particular when T is an epoxide, the functionalisation is carried out at a pH greater than or equal to 9, or greater than or equal to 10, more advantageously greater than or equal to 12, and in particular at a pH less than 14, for example less than or equal to 13.5. For this purpose, the reaction medium preferably comprises a Bronsted base, more preferably a hydroxide, yet more preferably sodium or potassium hydroxide. Advantageously, the reaction medium comprises sodium or potassium hydroxide at a concentration between 0.10 M and 0.30 M.

In certain embodiments, in particular when T is an amino group, the functionalisation is carried out at a pH less than 7, more advantageously greater than or equal to 4.5 and less than 7 or less than or equal to 6.5. For this purpose, the reaction medium preferably comprises a Bronsted acid, more preferably hydrochloric acid, sulfuric acid, or acetic acid.

The mass concentration of polysaccharide of the functionalisation reaction medium is advantageously between 50 and 300 mg/g of functionalisation medium, preferably between 100 and 200 mg/g.

In certain embodiments, the functionalisation of the polysaccharide is carried out at a temperature between 4° C. and 60° C., more preferably between 10° C. and 50° C., yet more preferably between 10° C. and 25° C. In these embodiments, the duration of the functionalisation reaction can vary from 1 hour to 2 weeks, more particularly from 3 hours to 1 week, yet more particularly from 3 hours to 96 hours, for example from 3 hours to 80 hours, in particular from 3 hours to 75 hours.

In certain embodiments, in particular when the functionalisation and crosslinking of the polysaccharide are concomitant or partially concomitant, the functionalisation of the polysaccharide can be at least partially carried out at a pressure P less than or equal to atmospheric pressure, and at a temperature T greater than the temperature of the eutectic point of the reaction medium as measured at pressure P, and less than the temperature of the freezing point of the reaction medium as measured at pressure P. The higher the functionalisation temperature, the shorter will be the functionalisation time in order to obtain the same degree of functionalisation.

### Step d)

The functionalised polysaccharide is crosslinked by sol-gel reaction in order to give a hydrogel.

This step enables the polysaccharide chains to be cross-linked with one another when they are functionalised with molecules of formula Chem. I. More specifically, during this step, at least some of the Si—OR¹⁰ groups and optionally at least some of the SiOR⁴ groups will react two-by-two, optionally after hydrolysis of these groups, to form Si—O—Si bonds. This implies that two molecules of formula Chem. I grafted on the polysaccharide chains will react together via their Si—OR¹⁰ (or SiOR⁴, as applicable) end groups and bond covalently via the formation of a Si—O—Si bond thus enabling bonding together of the polysaccharide chains and their crosslinking.

In this way, crosslinked polysaccharides are obtained, comprising crosslinking bonds between two polysaccharide chains, said crosslinking bonds comprising a divalent —Si—O—Si— group.

For this reason, step d) cannot take place before step c).

Step d) is carried out at least partially (in other words in part or entirely) at a pressure P less than or equal to atmospheric pressure, and at a temperature T greater than the temperature of the eutectic point of the reaction medium as measured at pressure P, and less than the temperature of the freezing point of the reaction medium as measured at pressure P. The duration of maintaining these conditions depends on the pH of the reaction medium. Thus when the pH of the reaction medium is greater than or equal to 9, or greater than or equal to 10, and less than 14, the pressure P and the temperature T are maintained for a duration t ranging from 2 to 17 weeks, in particular from 2 to 10 weeks or from 2 to 9 weeks, for example from 3 to 6 weeks, more advantageously for approximately 4 to 5 weeks. When the pH of the reaction medium is greater than or equal to 6.8 and less than or equal to 7.8, the pressure P and the temperature T are maintained for a duration t ranging from 1 to 48 hours, preferably greater than or equal to 6 hours and less than or equal to 36 hours, in particular greater than or equal to 7 hours and less than or equal to 36 hours.

It should be understood that the reaction conditions (pH, T, P) disclosed above can correspond to conditions applied throughout the entire duration of the crosslinking step (step d)) or may correspond to conditions applied for only a part of the duration of step d). In other words, the duration of crosslinking step d) can be greater than the durations t indicated above, the reaction conditions (pH or P or T) applied in the additional time then being different from those disclosed above.

The temperature of the freezing point of the reaction medium designates the temperature at which the mixture of the components of the reaction medium, on the macroscopic scale, solidify, in other words become non-fluid. Below the freezing point, the mixture is in a frozen state which is characterised by the coexistence of components in solid and liquid form. The freezing state is maintained down to the temperature of the eutectic point of the reaction medium.

The temperature of the eutectic point of the reaction medium designates the temperature below which the mixture of the components of the reaction medium passes from a frozen state (coexistence of liquid and solid phases) to a completely solid state, in other words a state in which all the components of the mixture are in solid form.

The freezing point and the eutectic point of a mixture depend on the pressure to which the mixture is subjected, therefore the freezing point and the eutectic point are measured at pressure P.

The freezing point and the eutectic point can be determined by differential scanning calorimetry. This method makes it possible to determine the phase transitions. For this purpose, the product to be studied is gradually cooled until its phase transitions are observed.

“Atmospheric pressure” is the pressure that is exerted by the air which constitutes the atmosphere on any surface in contact with it. It varies as a function of altitude. At an altitude of 0 m, the average air pressure is 101,325 Pa.

At temperature T and at pressure P, the reaction medium is thus frozen.

The sol-gel reaction typically takes place in an aqueous reaction medium.

The mass concentration of polysaccharide in the reaction medium is advantageously between 50 and 300 mg/g of sol-gel reaction medium, preferably between 100 and 200 mg/g.

Preferably, the pressure P is between 10⁻³ mbar and atmospheric pressure, more preferably, the pressure P is atmospheric pressure.

Preferably, the temperature T is greater than or equal to −55° C. and less than or equal to −5° C., preferably it ranges from −35° C. to −10° C., in particular from −30° C. to −10° C. or from −25° C. to −15° C. Yet more preferably, temperature T is approximately −20° C.

Preferably, the pressure P is atmospheric pressure and the temperature T is greater than or equal to −55° C. and less than or equal to −5° C., preferably it ranges from −35° C. to −10° C., in particular from −30° C. to −10° C. or from −25° C. to −15° C., more preferably, temperature T is approximately −20° C.

Advantageously, the reaction medium is placed and maintained at temperature T by contact of the container comprising the reaction medium with air or a liquid L at temperature T. The liquid L may be, in particular, ethylene glycol, glycerol or an azeotropic mixture of these with water. The liquid L will be chosen as a function of the desired temperature T so as to be liquid at this temperature T. More advantageously, the reaction medium is left at temperature T by contact of the content comprising the reaction medium with air at temperature T.

Typically, the lower the temperature T, the longer the duration t in order to obtain hydrogels having equivalent mechanical properties. Indeed, the lower the temperature T, the less kinetic is the sol-gel reaction.

Similarly, the lower the functionalisation ratio, the longer the duration t in order to obtain hydrogels having equivalent mechanical properties.

In other words, the lower the molar quantity of molecule of formula Chem. I or a salt thereof per 1 mole of polysaccharide repetition unit, the fewer are the SiOH functions in the reaction medium and the lower will be the probability that 2 groups meet and react together, thus the longer the duration t must be in order to enable the Si—OH functions to react with one another and to form crosslinking bonds, and thus to obtain a gel with desirable properties.

Thus, with a functionalisation ratio of 4 to 18%, the duration t is generally from 2 weeks to 15 weeks when the pH of the reaction medium is greater than or equal to 9, or greater than or equal to 10, and less than 14.

With a functionalisation ratio of 4 to 8%, the duration t is generally from 8 weeks to 15 weeks when the pH of the reaction medium is greater than or equal to 9, or greater than or equal to 10, and less than 14.

With a functionalisation ratio of 8 to 12%, the duration t is generally from 3 weeks to 10 weeks when the pH of the reaction medium is greater than or equal to 9, or greater than or equal to 10, and less than 14.

With a functionalisation ratio of 12 to 18%, the duration t is generally from 2 weeks to 6 weeks when the pH of the reaction medium is greater than or equal to 9, or greater than or equal to 10, and less than 14.

For a same molar quantity of molecule of formula Chem. I or a salt thereof per 1 mole of polysaccharide repetition unit, the lower the weight average molar mass Mw of the polysaccharide, the longer the duration t in order to obtain hydrogels having equivalent mechanical properties.

When the crosslinking by sol-gel reaction at temperature T and pressure P is carried out in a reaction medium at a pH greater than or equal to 9, or greater than or equal to 10, and less than 14, the reaction medium preferably comprises a Bronsted base, more preferably a hydroxide, yet more preferably sodium or potassium hydroxide. Advantageously, the reaction medium comprises sodium or potassium hydroxide at a concentration between 0.10 M and 0.30 M.

Preferably, at the end of the duration t (crosslinking carried out at pressure P and temperature T), the pH of the reaction medium is adjusted to a physiological pH, preferably to a pH of approximately 6.8 to 7.8. It should be understood that at the end of the duration t, the reaction medium is brought back to ambient temperature and placed or maintained at atmospheric pressure (if P is equal to atmospheric pressure).

When the crosslinking by sol-gel reaction at temperature T and pressure P is carried out in a reaction medium at physiological pH (pH greater than or equal to 6.8 and less than or equal to 7.8) and the functionalisation is carried out in a base medium (pH greater than or equal to 9, or greater than or equal to 10, and less than 14), the pH of the reaction medium will be brought to a physiological pH before the temperature is brought to temperature T and before the pressure is brought to pressure P if this is less than the atmospheric pressure. In this case, the method will advantageously comprise a neutralisation step of the gel in order to reach this physiological pH, before the reaction medium is brought to temperature T and pressure P. For this purpose, a Bronsted acid is preferably added to the reaction medium, preferably an aqueous solution of hydrochloric acid, an aqueous solution of sulfuric acid or an aqueous solution of acetic acid.

When the crosslinking by sol-gel reaction at temperature T and pressure P is carried out in a reaction medium at physiological pH (pH greater than or equal to 6.8 and less than or equal to 7.8) and the functionalisation is carried at a pH less than 7, for example greater than or equal to 4.5 and less than 7 or less than or equal to 6.5, the pH of the reaction medium will be brought to a physiological pH before the temperature is brought to temperature T and before the pressure is brought to pressure P if this is less than the atmospheric pressure.

### Steps c) and d), Concomitant or Partially Concomitant

Very generally, the method of the present invention comprises concomitantly or partially concomitantly carrying out steps c) and d). A concomitant carrying out of steps c) and d) makes it possible to shorten the duration of the method for preparing of the hydrogel and to simplify it.

The method of the present invention therefore comprises steps a) to d) as described above and is characterised in that steps c) and d) are concomitant or partially concomitant. The method then comprises:
functionalisation and crosslinking by sol-gel reaction carried out in a reaction medium at a pH greater than or equal to 9, or greater than or equal to 10, and less than 14, at a pressure P less than or equal to atmospheric pressure and at a temperature T greater than the temperature of the eutectic point of the reaction medium as measured at pressure P, and less than the temperature of the freezing point of the reaction medium as measured at pressure P, for a duration t ranging from 2 weeks to 17 weeks, in particular from 2 to 10 weeks or from 2 to 9 weeks, for example from 3 to 6 weeks, more advantageously approximately 4 to 5 weeks, or
a functionalisation and crosslinking by sol-gel reaction carried out in a reaction medium at a pH greater than or equal to 6.8 and less than or equal to 7.8, at pressure P less than or equal to atmospheric pressure and at a temperature T greater than the temperature of the eutectic point of the reaction medium as measured at pressure P, and less than the temperature of the freezing point of the reaction medium as measured at pressure P, for a duration t between 1 hour and 48 hours, preferably greater than or equal to 6 hours and less than or equal to 36 hours, in particular greater than or equal to 7 hours and less than or equal to 36 hours.

It should be understood from the above that steps c) and d) are at least partially (in part or in full) carried out under conditions disclosed above (T, P, pH, t).

In certain embodiments, the functionalisation of the polysaccharide and the crosslinking of the functionalised polysaccharide are then carried out as follows:
1) preparing a reaction medium comprising the one or more polysaccharides, the one or more molecules of formula Chem. I and a solvent, the pH of the reaction medium being greater than or equal to 9, or greater than or equal to 10, and less than 14;
2) optionally placing the reaction medium at a temperature ranging from 4° C. to 60° C., preferably from 10° C. to 50° C., more preferably from 10 to 25° C., typically for a duration ranging from 1 hour to 2 weeks, more particularly from 3 hours to 1 week, for example from 3 hours to 80 hours, in particular from 3 hours to 75 hours;
3) optionally adjusting the pH of the reaction medium to a pH greater than or equal to 6.8 and less than or equal to 7.8;
4) placing the reaction medium:
at a pressure P less than or equal to atmospheric pressure and at a temperature T greater than the temperature of the eutectic point of the reaction medium as measured at pressure P, and less than the temperature of the freezing point of the reaction medium as measured at pressure P, for a duration t ranging from 2 weeks to 17 weeks, when the pH of the reaction medium is greater than or equal to 9, greater than or equal to 10, and less than 14, in particular from 2 to 10 weeks or from 2 to 9 weeks, for example from 3 to 6 weeks, more advantageously approximately 4 to 5 weeks, or
at a pressure P less than or equal to atmospheric pressure and at a temperature T greater than the temperature of the eutectic point of the reaction medium as measured at pressure P, and less than the temperature of the freezing point of the reaction medium as measured at pressure P, for a duration t between 1 hour and 48 hours, preferably greater than or equal to 6 hours and less than or equal to 36 hours, in particular greater than or equal to 7 hours and less than or equal to 36 hours, when the pH of the reaction medium is greater than or equal to 6.8 and less than 7.8.

In other words, the method of the present invention can then be defined in the following manner:
a) providing at least one polysaccharide;
b) providing at least one molecule of formula Chem. I as described above;
c) functionalisation of the polysaccharide with at least one molecule of formula Chem. I as described above;
d) crosslinking by sol-gel reaction of the functionalised polysaccharide in order to give a hydrogel;
wherein the functionalisation and crosslinking of the functionalised polysaccharide are carried out as follows:
1) preparing a reaction medium comprising the one or more polysaccharides, the one or more molecules of formula Chem. I and a solvent, the pH of the reaction medium being greater than or equal to 9, or greater than or equal to 10, and less than 14;
2) optionally placing the reaction medium at a temperature ranging from 4° C. to 60° C., preferably from 10° C. to 50° C., more preferably from 10 to 25° C., typically for a duration ranging from 1 hour to 2 weeks, more particularly from 3 hours to 1 week, for example from 3 hours to 80 hours, in particular from 3 hours to 75 hours;
3) optionally adjusting the pH of the reaction medium to a pH greater than or equal to 6.8 and less than or equal to 7.8;
4) placing the reaction medium:
   at a pressure P less than or equal to atmospheric pressure and at a temperature T greater than the temperature of the eutectic point of the reaction medium as measured at pressure P, and less than the temperature of the freezing point of the reaction medium as measured at pressure P, for a duration t ranging from 2 weeks to 17 weeks, in particular from 2 to 10 weeks or from 2 to 9 weeks, for example from 3 to 6 weeks, more advantageously approximately 4 to 5 weeks, when the pH of the reaction medium is greater than or equal to 9, or greater than or equal to 10 and less than 14, or
   at a pressure P less than or equal to atmospheric pressure and at a temperature T greater than the temperature of the eutectic point of the reaction medium as measured at pressure P and less than the temperature of the freezing point of the reaction medium as measured at pressure P, for a duration t between 1 hour and 48 hours, preferably greater than or equal to 6 hours and less than or equal to 36 hours, in particular greater than or equal to 7 hours and less than or equal to 36 hours, when the pH of the reaction medium is greater than or equal to 6.8 and less than 7.8.

The solvent is typically water or a mixture comprising water and an organic solvent (for example an alcohol, in particular ethanol, or DMSO; typically a mixture comprising at least 90% by weight water, or at least 95% or at least 99% by weight water relative to the total weight of the solvent).

The reaction medium typically comprises 0.01 to 0.50, preferably 0.05 to 0.45, in particular 0.10 to 0.25 moles of the molecule of formula Chem. I or a salt thereof, per 1 mole of polysaccharide repetition unit.

The mass concentration of polysaccharide of the reaction medium is advantageously between 50 and 300 mg/g of solvent, preferably between 100 and 200 mg/g.

In certain embodiments, the functionalisation and the crosslinking are carried out in a reaction medium for which the pH is greater than or equal to 9, or greater than or equal to 10, and less than 14. For this purpose, the reaction medium preferably comprises a Bronsted base, more preferably a hydroxide, yet more preferably sodium or potassium hydroxide. Advantageously, the reaction medium comprises sodium or potassium hydroxide at a concentration between 0.10 M and 0.30 M.

In these embodiments, according to a first variant, the reaction medium prepared in step 1) can be placed at a temperature ranging from 4° C. to 60° C., preferably from 10° C. to 50° C., yet more preferably from 10° C. to 25° C., typically for a duration ranging from 1 hour to 2 weeks, more particularly from 3 hours to 1 week, for example from 3 hours to 80 hours, in particular from 3 hours to 75 hours (step 2) before being placed at temperature T and pressure P, for a duration t (step 4). During step 2), the polysaccharide will be functionalised and some of the Si—OR¹⁰ groups and optionally some of the Si—OR⁴ groups will condense with each other (pre-condensation), with more Si—OR¹⁰ groups and optionally some Si—OR⁴ groups condensing during step 4) (advance condensation).

In these embodiments, according to a second variant, the reaction medium prepared in step 1) can be placed directly at the end of step 1) at temperature T and pressure P for a duration t (step 4).

At the end of time t (first and second variant), the temperature of the reaction medium is typically returned to ambient temperature and the reaction medium placed at atmospheric pressure if pressure P is different from atmospheric pressure. The pH of the reaction medium is then preferably brought to a physiological pH (approximately 6.8 to 7.8). For this purpose, a Bronsted acid is preferably added to the reaction medium, preferably an aqueous solution of hydrochloric acid, an aqueous solution of sulfuric acid or an aqueous solution of acetic acid.

In certain embodiments, the crosslinking is carried out partially in a reaction medium, for which the pH is greater than or equal to 6.8 and less than 7.8. In these embodiments, a reaction medium having a pH greater than or equal to 9, or greater than or equal to 10, and less than 14 and comprising the one or more polysaccharides, the one or more molecules of formula Chem. I and a solvent is prepared (step 1)). The reaction medium is placed at a temperature ranging from 4° C. to 60° C., preferably from 10° C. to 50° C., yet more preferably from 10° C. to 25° C., typically for a duration ranging from 1 hour to 2 weeks, more particularly from 3 hours to 1 week, for example from 3 hours to 80 hours, in particular from 3 hours to 75 hours (step 2).

Then, the pH of the reaction medium is brought to a physiological pH (step 3)) before the temperature is brought to T before the pressure is brought to pressure P if this is less than atmospheric pressure. In this case, the method will advantageously comprise a neutralisation step of the gel in order to reach this physiological pH, before the reaction medium is brought to temperature T and pressure P. For this purpose, a Bronsted acid is preferably added to the reaction medium, preferably an aqueous solution of hydrochloric acid, an aqueous solution of sulfuric acid or an aqueous solution of acetic acid. The reaction medium is then placed at temperature T and pressure P for a duration t between 1 hour and 48 hours (step 4).

Preferably, the method of the present invention comprises only a single step of placing the reaction medium at temperature T and pressure P.

### Step e)

The method according to the invention can comprise an additional step e) of crosslinking the polysaccharide with a conventional crosslinking agent, and more particularly crosslinking of the polysaccharide provided in step a) or of the crosslinked polysaccharide obtained following step d), in the presence of at least one crosslinking agent or of a salt thereof, said crosslinking agent comprising at least two functional groups Z as described below.

The “crosslinking agent” or “agent for crosslinking” is a compound comprising at least two functional groups which are able to covalently bond with functional groups present on the polysaccharide, such as OH, CHO, NH₂ or COOH groups carried by the polysaccharide, and thus to induce bonds between the polysaccharide chains (crosslinking) and/or bonds on a same polysaccharide chain.

The crosslinking agent used in the context of the present invention comprises at least two, preferably 2 to 8, in particular 2, functional groups (designated “Z groups”) preferably independently chosen from isocyanate (—N═C═O), amino (—NH₂), epoxide, carboxyl (—COOH), N-succinimidyloxycarbonyl, N-sulfosuccinimidyloxycarbonyl, halogenocarbonyl, isothiocyanate (—N═C═S), vinyl (—CH═CH₂), formyl (—CH═O), hydroxyl (—OH), sulfhydryl (—SH), hydrazino (—NH—NH₂), acylhydrazino (—CO—NH—NH₂), aminoxy (—O—NH₂), and carbodiimide groups, and an acid anhydride residue. The functional groups are preferably identical.

The isocyanate can react with an OH or NH₂ group of the polysaccharide to form a carbamate or urea function. The amino group can react with a COOH group of the polysaccharide to form an amide function. The epoxide group can react with an OH or COOH group of the polysaccharide to form an ether or ester function. The carboxyl group can react with an OH or NH₂ group of the polysaccharide to form an ester or amide function. The N-succinimidyloxycarbonyl and N-sulfosuccinimidyloxycarbonyl groups can react with an OH or NH₂ group of the polysaccharide to form an ester or amide function. The halogenocarbonyl can react with an OH or NH₂ group of the polysaccharide to form an ester or amide function. The isothiocyanate group can react with an OH or NH₂ group of the polysaccharide to form a thiocarbamate or thiourea function. The vinyl group can react with an OH group of the polysaccharide to form an ether function. The formyl group can react with an OH or NH₂ group of the polysaccharide to form a hemiacetal or hemiaminal function.

The hydroxyl group can react with a COOH group of the polysaccharide to form an ester function. The sulfhydryl group can react with a COOH group of the polysaccharide to form a thioester function.

The hydrazino (—NH—NH₂) group can react with a CHO group of the polysaccharide to form a hydrazone function. The acylhydrazino group can react with a CHO group of the polysaccharide to form a carbonyl hydrazone ═NNHC(O)— group. The aminoxy group can react with a CHO group of the polysaccharide to form an oxime ═NO— function. The carbodiimide group can react with a COOH group of the polysaccharide to give a CO—NR^{a}—CO—NH function, and an acid anhydride residue can react with an OH or NH₂ group of the polysaccharide to form an ester or amide function.

In particular, the group T of the molecule of formula Chem. I and the groups Z are identical.

Preferably, the functional groups Z are identical and represent an epoxide or vinyl group, more preferably epoxide.

According to another advantageous embodiment, the functional groups Z are identical and chosen from the amino, vinyl, formyl, and carbodiimide groups, are preferably amino groups.

In particular, the crosslinking agent is chosen from hexamethylene diisocyanate, 4,4′-diphenylmethylene diisocyanate, 4-arm PEG20K-isocyanate, spermine (or 1,12-diamino-5,9-diazadodecane), spermidine (or 1,8-diamino-5-azaoctane), cadaverine (or 1,5-diaminopentane), putrescine (1,4-diaminobutane), poly(ethylene glycol) diamine, ethylenediamine, 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end by a diglycidyl ether (CAS number: 130167-23-6), poly(ethylene glycol) diacid, disuccinimidyl suberate, bis(sulfosuccinimidyl)suberate, sebacoyl chloride, 1,4-butane diisothiocyanate, divinyl sulfone (DVS), glutaraldehyde, polyethylene glycol, 1,5-pentanedithiol, adipic acid dihydrazide, bis-aminooxy-poly(ethylene glycol), diethylenetriaminepentaacetic acid dianhydride, and the mixtures thereof.

When the functional groups Z are epoxide groups, the crosslinking agent is preferably chosen from 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), 1,3-bis(3-glycidyloxypropyl)tetramethyldisiloxane, poly(dimethylsiloxane) terminated at each end by a diglycidyl ether (CAS number: 130167-23-6), hydroxyapatite beads modified to carry epoxy groups and the mixtures thereof.

More preferably, the crosslinking agent is chosen from 1,4-butanediol diglycidyl ether (BDDE), 1,2,7,8-diepoxy-octane, poly(ethylene glycol) diglycidyl ether (PEGDGE), 1,2-bis(2,3-epoxypropoxy)ethane (EGDGE), and the mixtures thereof.

When the functional groups Z are amino groups, the crosslinking agent is preferably a polyamine chosen from spermine (or 1,12-diamino-5,9-diazadodecane), spermidine (or 1,8-diamino-5-azaoctane), cadaverine (or 1,5-diaminopentane), putrescine (or 1,4-diaminobutane), their salts or a mixture thereof, more preferably the crosslinking agent is a polyamine chosen from spermine, spermidine, their salts and the mixtures thereof. When the functional groups Z are amino groups, the crosslinking reaction of step e) with the polysaccharide is advantageously carried out in the presence of at least one activator, and where appropriate combined with at least one coupling auxiliary.

In this respect, the activator can be selected from the water-soluble carbodiimides, such as 1-ethyl-3-(3-dimethylaminopropyl)carbodiimide (EDC), 1-ethyl-3-[3-(trimethylamino)propyl]carbodiimide hydrochloride (ETC), the 1-cyclohexyl-3-(2-morphilinoethyl)carbodiimide (CMC), their salts and the mixtures thereof, is preferably EDC.

With respect to the coupling auxiliary, when it is present, it can be selected from N-hydroxy succinimide (NHS), N-hydroxybenzotriazole (HOBt), 3,4-dihydro-3-hydroxy-4-oxo-1,2,3-benzotriazole (HOOBt), 1-hydroxy-7-7azabenzotriazole (HAt) and N-hydroxysylfosuccinimide (sulfo NHS), and the mixtures thereof, is preferably HOBt.

The crosslinking agent can be chosen from hydroxyapatite beads modified to carry epoxy groups, a compound of formula Chem. II as described below, and the mixtures thereof.

Preferably, the crosslinking agent is a compound of formula Chem. II:

Y—(Z)ₙ

wherein the functional groups Z, identical or different, are as defined above,
n is an integer greater than or equal to 2, in particular ranging from 2 to 8, preferably equal to 2,
Y is an, in particular aliphatic, polyvalent hydrocarbon group, having a valence of n and comprising 1 to 150 carbon atoms:
   wherein one or more (for example 1 to 150, or even 1 to 50 or even to 15 or even 1 or 2) CH₂ units are optionally replaced by one or more divalent units chosen from the arylenes; —O—; —S—; —S(O)—; —C(═O)—; —SO₂—; —N(R¹)—; and —[SiR²R³O]ₘ—SiR²R³—
where
R¹ representing a hydrogen atom, an aliphatic hydrocarbon group having 1 to 6 carbon atoms, or an aryl-(C1-C6)alkyl;
m is an integer between 1 and 20; and
R² and R³, identical or different, represent a hydrogen atom; a halogen atom; an —OR″ group with R″ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl,
   said polyvalent group being non-substituted or substituted by one or more monovalent groups chosen from a halogen atom, a hydroxyl, an aryl-(C1-C6)alkyl, preferably non-substituted.

In particular, n is an integer ranging from 2 to 8, preferably n represents 2, 3 or 4, more preferably n is equal to 2.

Advantageously, R¹ represents a hydrogen atom or a (C1-C6)alkyl group.

In particular, R² and R³, identical or different, represent an aliphatic hydrocarbon group having 1 to 6 carbon atoms, more particularly a (C1-C6)alkyl group.

Preferably, in the definition of Y, the polyvalent hydrocarbon group can be a polyvalent aliphatic or aromatic hydrocarbon group, preferably aliphatic and in particular saturated, having a valence of n and having 1 to 150 carbon atoms, preferably 1 to 50 carbon atoms, more preferably 1 to 20 carbon atoms, yet more preferably 2 to 20 carbon atoms. In particular, in the definition of Y, the polyvalent group hydrocarbon is a saturated, in particular linear, polyvalent aliphatic hydrocarbon group.

Preferably, Y is a polyvalent hydrocarbon group as described above, wherein one or more CH₂ units are optionally replaced by one or more divalent units chosen from —O—, —SO₂—, —[SiR²R³O]ₘ—SiR²R³— and —NH—, with R², R³ and m as described above.

In particular, Y is a polyvalent hydrocarbon group as described above, preferably aliphatic unsaturated, and in particular linear, branched, or star-shaped, and optionally wherein:
at least two CH₂ units replaced by —O—, in particular between 1 and 50 CH₂ units, more particularly between 1 and 15 CH₂ units, or
at least one, preferably one or two, CH₂ unit is replaced by an —NH— unit, or
at least one, preferably one, CH₂ unit is replaced by an —SO₂— unit, or
at least two, preferably two, CH₂ units are replaced by —O— and at least one, preferably one, CH₂ unit is replaced by an —[SiR²R³O]ₘ—SiR²R³— unit with R², R³ and m as described above.

More particularly, when one or more CH₂ units are replaced by —O—, the one or more units replaced are such as Y comprises one or more —CH₂—CH₂—O— units. In particular, Y comprises 1 to 50 —CH₂—CH₂—O— units, advantageously 2 to 25 —CH₂—CH₂—O— units, more advantageously 2 to 15 —CH₂—CH₂—O— units. Y may comprise only —CH₂—CH₂—O— units. More preferably, Y is a preferably linear alkyl group comprising 1 to 150, in particular 1 to 50, in particular 1 to 20, for example 1 to 12, in particular 1 to 6 carbon atoms, wherein optionally one or more CH₂ units are replaced by one or more divalent units chosen from —O— and —NH—, more particularly between 1 and 50, in particular between 1 and 15, for example 1 or 2, divalent units chosen from —O— and —NH—.

According to a first embodiment, R² and R³, identical or different, represent an —OR″ group with R″ as described above. In particular, R″ represents an aliphatic hydrocarbon group having 1 to 6 carbon atoms, more particularly a (C1-C6)alkyl group.

According to a second embodiment, R² and R³, identical or different, represent an aliphatic hydrocarbon group having 1 to 6 carbon atoms optionally substituted (preferably non-substituted) by one or more groups chosen from a halogen atom, an aryl or a hydroxyl, more preferably a non-substituted (C1-C6)alkyl groups such as a methyl or ethyl.

Advantageously, the crosslinking agent is a compound of following formula Chem. Ila:

Z¹—Y¹—Z²

wherein the Z¹ and Z² groups, identical or different, are chosen from the isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy and carbodiimide groups, and an acid anhydride residue, and Y¹ represents a divalent, in particular aliphatic, hydrocarbon chain having 1 to 50 carbon atoms:
wherein one or more (for example 1 to 15, or even 1 or 2) CH₂ units are optionally replaced by one or more divalent units chosen from the arylenes; —O—; —S—; —S(O)—; —C(═O)—; —SO₂—; —N(R¹)—; and —[SiR²R³O]ₘ—SiR²R³— with:
R¹ representing a hydrogen atom, an aliphatic hydrocarbon group having 1 to 6 carbon atoms, or an aryl-(C1-C6),
m is an integer between 2 and 20; and
R² and R³, identical or different, represent a hydrogen atom, halogen atom; an —OR″ group with R″ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl,
   said chain being non-substituted or substituted by one or more monovalent groups chosen from a halogen atom, a hydroxyl, an aryl-(C1-C6)alkyl group.

The Z¹ and Z² groups have the same definition as the Z group defined above.

In particular, the group T of the molecule of formula Chem. I and the Z¹ and Z² groups of the molecule of formula Chem. Ila are identical.

Y¹ have the same definition as Y defined above with a valence not being equal to 2.

In particular Y¹ may comprise only —CH₂—CH₂—O— units, as previously defined.

Preferably, the crosslinking agent of formula Chem. II or Chem. Ila does not comprise —[SiR²R³O]ₘ—SiR²R³— units.

The crosslinking of step e) of the polysaccharide provided in step a) or of the polysaccharide obtaining following step d) takes place preferably in the presence of 0.05 to 10 moles, in particular 0.05 to 7 moles, more advantageously 0.05 to 5 moles, yet more advantageously 0.1 to 2 moles, of at least one crosslinking agent per 100 moles of polysaccharide repetition unit. In certain embodiments, the crosslinking of step e) of the polysaccharide provided in step a) or of the polysaccharide obtained following step d) takes place in the presence of 0.1 to less than 2 moles, or 0.1 to 1.5 moles or 0.1 to 1 mole or 0.1 to 0.8 moles or 0.1 to 0.5 moles of at least one crosslinking agent per 100 moles of polysaccharide repetition unit

In particular, the crosslinking of step e) takes place in aqueous reaction medium.

However, if necessary, an organic solvent such as an alcohol, in particular ethanol, or DMSO, can be used to solubilise the crosslinking agent, for example when it involves poly(dimethylsiloxane) terminated at each end by a diglycidyl ether (CAS number: 130167-23-6) before addition to the aqueous reaction medium.

Advantageously, and in particular when the groups Z, such as Z¹ or Z², represent an epoxide group or a vinyl group, the crosslinking of step e) takes place at a pH greater than or equal to 10, more advantageously greater than or equal to 12.

For this purpose, the reaction medium preferably comprises a Bronsted base, more preferably a hydroxide salt, such as sodium or potassium hydroxide. In particular, the reaction medium comprises a Bronsted base, more preferably a hydroxide, yet more preferably sodium or potassium hydroxide at a concentration between 0.10 M and 0.30 M.

According to an embodiment, the crosslinking of step e) takes place between 4° C. and 60° C., more preferably between 10° C. and 50° C.

In particular, the crosslinking of step e) takes place between 1 hour and 2 weeks, more particularly between 3 hours and 1 week.

According to a variant, the crosslinking of step e) takes place at temperature T and pressure P, for a duration t as described above, the duration t varying as a function of the pH of the reaction medium. In this case, step e) is advantageously concomitant at least in part or totally with step d) and optionally step c).

In the presence of a plurality of crosslinking agents, the crosslinking agents can be added simultaneously or at separate times. Step e) can thus comprise repeated crosslinking steps. The total quantity of crosslinking agents varies from 0.05 to 10 moles, preferably from 0.05 to 7 moles, more advantageously from 0.05 to 5 moles, yet more advantageously from 0.1 to 2 moles, per 100 moles of polysaccharide repetition unit. In certain embodiments, the total quantity of crosslinking agents varies from 0.1 to less than 2 moles or 0.1 to 1.5 moles or 0.1 to 1 mole or 0.1 to 0.8 moles or 0.1 to 0.5 moles of crosslinking agents per 100 moles of polysaccharide repetition unit.

This step enables crosslinking of the polysaccharide chains with one another.

The functional groups of the crosslinking agent react with functional groups present on the polysaccharides so as to bond the polysaccharide chains to one another and to crosslink them by forming intermolecular bonds. The crosslinking agent can also react with the functional groups present on a same polysaccharide molecule so as to form intramolecular bonds. Notably, the functional groups of the crosslinking agent react with —OH or —COOH, or optionally —CHO groups present on the polysaccharides, such as hyaluronic acid. Crosslinked polysaccharides are thus obtained, comprising at least one crosslinking bond between two polysaccharide chains, said crosslinking bond being the residue of the crosslinking agent of step e)

In particular, following step e), the cross-linked polysaccharides comprise at least one crosslinking bond between two polysaccharide chains, said crosslinking bond comprising more particularly the polyvalent group Y as described above, preferably, the divalent¹ group Y as described above.

Certain functional groups Z (such as Z¹ and Z²) of the crosslinking agent may however not react with a polysaccharide chain.

In particular, when the crosslinking agent includes two functional groups Z¹ and Z², one of the Z¹ functional groups can react with a polysaccharide while the other Z² functional group does not react with any polysaccharide. A dangling bond is then formed.

In general, the quantity of functionalisation agent increases when the quantity of crosslinking agent is reduced, and vice versa.

According to a first variant, step e) takes place before step c), in particular between step a) and step c).

According to a second variant, steps c) and e) are concomitant. According to this variant, step d) can be at least partially concomitant with step c) as described above.

According to a third variant, step e) takes place after step c) and before d) or after step c) and at least partially concomitant with step d).

Thus, during step e), the reaction medium can be partially maintained preferably at temperature T and pressure P for a duration t as described above, this duration t varying as a function of the pH of the reaction medium, in particular when step e) is at least partially concomitant with step d).

According to a fourth variant, step e) takes place after step d).

In certain embodiments, step e) is not implemented.

### Step f)

Preferably, the method according to the invention further comprises a step f) of adding a molecule of the following formula Chem. Ill:

R⁷O—[R¹²R¹³SiO]ₚ—R³
or a salt thereof
wherein:
   p is an integer from 1 to 20;
R¹² and R¹³, identical or different, represent a hydrogen atom; a halogen atom; an —OR¹⁴ group with R¹⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl; and
   R⁷ and R⁸, identical or different, represent a hydrogen atom, an aryl group or an aliphatic hydrocarbon group having 1 to 6 carbon atoms.

According to an embodiment, step f) is carried out before the temperature of the reaction medium is at temperature T and pressure P during step d), in particular before, during or after step c).

According to a variant, step f) is carried out after step d), in other words after the reaction medium has been maintained at temperature T and pressure P.

When step c) and step d) are concomitant, step f) is carried out before these steps c) and d), in particular between step a) and step c).

Preferably, R¹² and R¹³, identical or different, represent an —OR¹⁴ group with R¹⁴ representing a hydrogen atom, an aryl group or an aliphatic hydrocarbon group having 1 to 6 carbon atoms; an aryl. an aliphatic hydrocarbon group having 1 to 6 carbon atoms optionally substituted by one or more groups chosen from a halogen atom, an aryl and a hydroxyl.

In particular, R¹² and R¹³, identical or different, represent an —OR¹⁴ group with R¹⁴ representing a hydrogen atom, an aliphatic hydrocarbon group having 1 to 6 carbon atoms, preferably a (C1-C6)alkyl group; or an aliphatic hydrocarbon group having 1 to 6 carbon atoms, preferably a (C1-C6)alkyl group.

Advantageously, R⁷ and R⁸, identical or different, represent a hydrogen atom, or an aliphatic hydrocarbon group having 1 to 6 carbon atoms, preferably a (C1-C6)alkyl group.

This molecule of formula Chem. Ill comprises Si—OR (Si—OR⁷, Si—OR⁸ groups and optionally Si—OR¹⁴) capable of reacting with the Si—OR (Si—OR¹⁰ groups and optionally Si—OR⁴) of the molecule of formula Chem. I. Thus, during the sol-gel reaction enabling the formation of Si—O—Si bonds, a molecule of formula Chem. Ill can bond to two molecules of formula Chem. I grafted on polysaccharide chains so as to form crosslinking bonds resulting from the coupling of a molecule of formula Chem. Ill with two molecules of formula Chem. I.

For example, the molecule of formula Chem. Ill is orthosilicic acid, tetraethyl orthosilicate (TEOS), polydimethylsiloxane (PDMS), oligomerised TEOS/orthosilicic acid, or methyl silanetriol (preferably used in the form of its sodium salt called sodium methyl siliconate—NAMS).

Advantageously, step f) takes place at a pH greater than or equal to 9, in particular greater than or equal to 10, more advantageously greater than or equal to 12, and in particular less than 14, for example less than or equal to 13.5, in particular when the molecule of formula Chem. Ill is sodium methyl siliconate (NAMS).

For this purpose, the reaction medium preferably comprises a Bronsted base, more preferably a hydroxide, yet more preferably sodium or potassium hydroxide. In particular, the reaction medium comprises a Bronsted base, more preferably a hydroxide, yet more preferably sodium or potassium hydroxide at a concentration between 0.10 M and 0.30 M.

Steps g) to i) which follow are gel formulation steps, carried out:
either all before the reaction medium is at temperature T and pressure P during step d),
or all after step d), in other words after the reaction medium has been maintained at temperature T and pressure P.

### Step g)

Advantageously, the method according to the invention further comprises a step g) of adding at least one additional component chosen from lubricants; cosmetic active ingredients such as antioxidants, co-enzymes, amino acids, vitamins, minerals, and nucleic acids; and the mixtures thereof, as described below.

This step g) may also comprise the addition of at least one therapeutic active ingredient advantageously chosen from anaesthetics, antibiotics, antifungals, adrenaline and its derivatives, and the mixtures thereof, as described below, in addition to or instead of the at least one additional component mentioned above.

Where applicable, step g) preferably takes place after the purification step h).

Where applicable, step g) preferably takes place before the sterilisation step j).

In particular, step g) can also comprise adding at least one therapeutic activity ingredient, or at least one cosmetic active ingredient, or the mixture thereof.

When at least one therapeutic active ingredient and/or at least one cosmetic active ingredient is added, step g) takes place preferably before the reaction medium is at temperature T and pressure P during step d).

### Step h)

The method preferably further comprises at least one step h) of purification, in particular by dialysis.

Where applicable, the at least one step h) preferably takes place before step g).

Where applicable, the at least one step h) preferably takes place before sterilisation step j).

Where applicable, the at least one step h) preferably takes place before sieving step i). According to a particular embodiment, a purification is carried out after step c). According to another embodiment, a purification is carried out after step e). According to another embodiment, a purification is carried out after step d).

### Step i)

In particular, the method further comprises a step i) of sieving, more particularly with a sieve having a porosity between 50 and 2000 μm.

This sieving step makes it possible to obtain a more homogeneous hydrogel with the most constant possible extrusion force, i.e. the most regular possible extrusion force. A person skilled in the art knows to select a sieve with suitable pore size depending on the mechanical properties of the hydrogel that it is desired to obtain. Step i) preferably takes place after steps a) to d) and optionally steps e), f), g) and h). Where applicable, step i) preferably takes place before sterilisation step j).

### Step j)

The method according to the invention can comprise a sterilisation step. The sterilisation is preferably carried out by heat, in particular by autoclave. The sterilisation is generally carried out by increasing the temperature of the sterilisation medium up to a so-called “plateau temperature”, which is maintained for a determined so-called “plateau duration”. The sterilisation is preferably carried out at a plateau temperature ranging from 121° C. to 135° C., preferably for a plateau duration ranging from 1 minute to 20 minutes with F0≥15. The sterilising value F0 corresponds to the time necessary, in minutes, at 121° C., to inactivate 90% of the population of microorganisms present in the product to be sterilised. Alternatively, the sterilisation can be carried out, in particular, by radiation with gamma rays, UV or by means of ethylene oxide.

Thus, advantageously, the method further comprises a step j) of sterilisation of the hydrogel, in particular a step of sterilisation by heat carried out at a plateau temperature between 121° C. and 135° C., preferably for a plateau duration between 1 minute and 20 minutes with F0 15 or a step of sterilisation by UV radiation.

According to a first alternative, the sterilisation step is carried out after steps a) to d) and the optional steps e), f), g), h) and i).

According to a second alternative, the sterilisation step takes place after step c) and before the reaction medium is at temperature T and pressure P during step d).

In this case, the pH of the reaction medium is adjusted to a physiological pH (6.8-7.8) before the sterilisation.

In particular, the gel is sterilised after having been packaged in its injection device and the packaging of the hydrogel takes place following all the steps of the method and before sterilisation.

### Step k)

In particular, the method further comprises a step k) of swelling the gel.

During the step of swelling the gel, the concentration of polysaccharide of the gel is adjusted. In particular, a solvent is added, for example water, a phosphate buffer, water for an injectable preparation. More particularly, the added solvent has a pH that is approximately the physiological pH.

The concentration of polysaccharide obtained following step k) is advantageously between 1 mg/g of gel and 50 mg/g of gel, more advantageously between 5 mg/g of gel and 35 mg/g of gel, yet more advantageously between 10 mg/g and 30 mg/g of gel.

Preferably, step k) takes place after step d) and the return of the reaction medium to a physiological pH (6.8-7.8), where applicable.

### Hydrogel

Another object of the present invention is a hydrogel which can be obtained by the method of the present invention. Such a hydrogel may also be designated by the term “cryogel”.

The liquid medium of the hydrogel is preferably an aqueous medium chosen from an aqueous solution or a mixture of aqueous solutions, preferably chosen from water for injectable preparation, phosphate saline buffer or a mixture of the two, more preferably phosphate saline buffer in the context of therapeutic, cosmetic and aesthetic applications according to the invention.

This hydrogel is preferably an injectable hydrogel. It is preferably sterile, in particular sterilised by heat at a plateau temperature between 121° C. and 135° C., preferably for a plateau duration between 1 minute and 20 minutes, with F0 15. The hydrogel is preferably homogeneous. The hydrogel is preferably stretchy, with in particular a phase angle δ of between 20° and 45°.

This hydrogel may also comprise an additional component chosen from lubricants; cosmetic active ingredients such as antioxidants, co-enzymes, amino acids, vitamins, minerals, and nucleic acids; and the mixtures thereof, as described below.

This hydrogel may also comprise at least one therapeutic active ingredient advantageously chosen from anaesthetics, antibiotics, antifungals, adrenaline and its derivatives, and the mixtures thereof, as described below.

The polysaccharide of this hydrogel is preferably as defined above, in the context of the description of step a) of the method according to the invention.

Preferably, a hydrogel according to the present invention, acceptable for the therapeutic and/or cosmetic applications targeted by the present invention, has a cross-over stress (or stress at the intersection of the G′ and G″ moduli) greater than or equal to 50 Pa, preferably between 50 and 5000 Pa and more preferably between 100 and 1000 Pa and an elastic modulus G′ greater than or equal to 20 Pa, preferably from 100 Pa to 2000 Pa, more preferably from 100 Pa to 1000 Pa.

Preferably, a hydrogel according to the present invention, acceptable for the therapeutic and/or cosmetic applications targeted by the present invention, has a cohesiveness of 1 N to 30 N. This cohesiveness is measured by mechanical compression using a rheometer. For this purpose, the gel is deposited on a peltier plane with an initial gap of 2.60 mm; it is then compressed at constant speed of 100 μm/s to 70% of the initial gap, at 25° C.; finally, the cohesiveness of the gel is measured at the end of the compression stroke. The more cohesive a gel is, i.e. the higher its value of cohesiveness, the more it is able to withstand stresses, such as those it may encounter after being administered to a.

### Composition

Another object of the present invention is a composition comprising the hydrogel according to the present invention. It preferably involves a cosmetic or pharmaceutical composition. It may further comprise physiologically acceptable excipients.

The hydrogel according to the invention comprises a crosslinked polysaccharide, preferably hyaluronic acid. The composition may further comprise a non-crosslinked polysaccharide, preferably hyaluronic acid.

The non-crosslinked hyaluronic acid can be present in the composition as a lubricant. The hydrogel according to the present invention can thus comprise 0.1 to 5% by weight, preferably 1 to 3% by weight polysaccharide, preferably hyaluronic acid, relative to the total weight of said composition, the polysaccharide such as hyaluronic acid, being present in crosslinked and optionally non-crosslinked form. In particular, the percentage of non-crosslinked polysaccharide, in particular hyaluronic acid, varies from 0 to 40% by weight, preferably from 1 to 40% by weight, more preferably from 5 to 30% by weight, relative to the total weight of polysaccharide, in particular hyaluronic acid, present in the composition.

The composition according to the present invention is preferably a sterile composition, in particular sterilised by heat at a plateau temperature between 121° C. and 135° C., preferably for a plateau duration between 1 minute and 20 minutes, with F0≥15. It is preferably an injectable composition. The composition according to the invention therefore preferably comprises a physiologically acceptable medium, preferably a physiologically acceptable aqueous medium.

The physiologically acceptable aqueous medium may comprise a solvent or a mixture of physiologically acceptable solvents and preferably comprises water, preferably the solvent is water.

The physiologically acceptable medium may also comprise isotonic agents such as oses, sodium chloride and the mixtures thereof.

The physiologically acceptable medium may further comprise at least one isotonic and physiologically acceptable saline solution.

Said balanced saline solution is preferably a phosphate buffered saline solution, and in particular a KH₂PO₄/K₂HPO₄ buffered saline solution.

The composition according to the invention may further comprise at least one additional compound chosen from lubricants; cosmetic active ingredients such as antioxidants, co-enzymes, amino acids, vitamins, minerals, and nucleic acids; and the mixtures thereof.

Preferably, the additional compound is water-soluble or modified to be soluble in an aqueous medium.

The composition according to the invention may also comprise at least one therapeutic active ingredient advantageously chosen from anaesthetics, antibiotics, antifungals, adrenaline and its derivatives, and the mixtures thereof, as described below. The therapeutic active ingredient is preferably water-soluble.

Examples of anaesthetics include ambucaine, the amoxecaine, amyleine, aprindine, aptocaine, articaine, benzocaine, betoxycaine, bupivacaine, butacaine, butamben, butanilicaine, chlorobutanol, chloroprocaine, cinchocaine, clodacaine, cocaine, cryofluorane, cyclomethycaine, dexivacaine, diamocaine, diperodon, dyclonine, etidocaine, euprocine, febuverine, fomocaine, guafecainol, heptacaine, hexylcaine, hydroxyprocaine, hydroxytetracaine, isobutamben, leucinocaine, levobupivacaine, levoxadrol, lidamidine, lidocaine, lotucaine, menglytate, mepivacaine, meprylcaine, myrtecaine, octacaine, octodrine, oxetacaine, oxybuprocaine, parethoxycaine, paridocaine, phenacaine, piperocaine, piridocaine, polidocanol, pramocaine, prilocaine, procaine, propanocaine, propipocaine, propoxycaine, proxymetacaine, pyrrocaine, quatacaine, quinisocaine, risocaine, rodocaine, ropivacaine, tetracaine, tolycaine, trimecaine or one of the salts thereof, in particular a hydrochloride thereof, and a mixture of these.

Examples of antioxidants include, in a non-limiting manner, glutathione, reduced glutathione, ellagic acid, spermine, resveratrol, retinol, L-carnitine, polyols, polyphenols, flavonols, theaflavins, catechins, caffeine, ubiquinol, ubiquinone, alpha-lipoic acid and their derivatives, and a mixture of these.

Examples of amino acids include, in a non-limiting manner, arginine (e.g., L-arginine), isoleucine (e.g., L-isoleucine), leucine (e.g., L-leucine), lysine (e.g., L-lysine or monohydrated L-lysine), glycine, valine (e.g., L-valine), threonine (e.g., L-threonine), proline (e.g., L-proline), methionine, histidine, phenylalanine, tryptophan, cysteine, their derivatives (e.g., N-acetyl derivatives such as N-acetyl-L-cysteine) and a mixture thereof. Examples of vitamins and their salts include, in a non-limiting manner, vitamins E, A, C, B, especially vitamins B6, B8, B4, B5, B9, B7, B12, and preferably pyridoxine and its derivatives and/or salts, preferably pyridoxine hydrochloride.

Examples of minerals include, in a non-limiting manner, the salts of zinc (e.g., zinc acetate, in particular dehydrated zinc acetate), magnesium salts, calcium salts (e.g., hydroxyapatite, in particular in bead form), potassium salts, manganese salts, sodium salts, copper salts (e.g., copper sulfate, in particular pentahydrated copper sulfate), optionally in a dehydrated form, and the mixtures thereof.

Nucleic acids include, in particular, adenosine, cytidine, guanosine, thymidine, cytosine, their derivatives and a mixture thereof.

Co-enzymes include the coenzyme Q10, CoA, NAD, NADP and the mixtures thereof. Derivatives of adrenaline include noradrenaline.

The quantities of additional compounds depend of course on the nature of the compound in question, the desired effect, and the destination of the composition as described here.

### Applications

The hydrogel or the composition according to the invention can have therapeutic, cosmetic or aesthetic applications.

The present invention therefore also relates to a hydrogel or a composition according to the invention for its use in the filling and/or replacement of tissues, in particular soft tissues, in particular by injection of the hydrogel or the composition into the tissue.

The hydrogel or the composition may be intended for superficial application.

A superficial application refers to the administration of a composition in the upper layers of the skin, i.e. in or on the skin, for example by mesotherapy and, for example, for reducing superficial wrinkles and/or for improving the quality of the skin (such as its radiance, density or structure) and/or rejuvenating the skin.

The hydrogel or the composition may be intended for a deep application.

A “deep application” refers to the administration of a composition in the deepest layers of the skin and/or under the skin (above the periosteum) in order to increase the volume of the soft tissues, such as for filling deep wrinkles and/or partially atrophied regions of the face and/or body.

The hydrogel or the composition can be versatile, i.e., can be used for both deep and superficial applications.

Preferably, when the hydrogel or the composition according to the invention comprises at least one therapeutic active ingredient, the present invention relates to the hydrogel or a composition according to the invention for its use in the modified, delayed or prolonged release of therapeutic active ingredients.

In particular, the hydrogel or the composition according to the invention is used in oral healthcare and more particularly in the treatment of gingival recession, or for filling periodontal pockets. More particularly, the hydrogel or the composition according to the invention is used for treating defects in the gingival architecture which can occur with tooth loss, with ageing, with periodontal diseases and disorders, or after the insertion of tooth implants, crowns or bridges.

The hydrogel or the composition according to the invention can also be used in ophthalmology, more particularly for protecting the ocular structures during eye surgery, for example ophthalmic surgery of the anterior or posterior segment, the removal of cataracts optionally with implantation of an intraocular lens, cornea transplant surgery, glaucoma filtering surgery, or implantation of a secondary lens. In this case, the hydrogel or the composition according to the invention will be more particularly injected into the eye.

The hydrogel or the composition according to the invention can also be used in orthopaedics or rheumatology, for example by injection into the synovial cavity. The hydrogel or the composition according to the invention is then used as a viscosupplementation.

The hydrogel or the composition according to the invention can also be used in the treatment of lipodystrophy.

The hydrogel or the composition according to the invention can be used in aesthetic surgery, in particular for gynecoplastias and/or penoplastias.

The hydrogel or the composition according to the invention is administered, more particularly, by injection.

The hydrogel or the composition according to the invention can also be used for the modified, delayed or prolonged release of therapeutic active ingredients, in particular the therapeutic active ingredients as described above. The longer the hydrogel is left at temperature T° and pressure P in the presence of the one or more active ingredients, the greater will be the sol-gel reaction and the slower will be the release of the active ingredient. Thus, it is possible to adapt the duration and/or the intensity of release of the active ingredient to the requirement. This also applies to the modified, delayed or prolonged release of cosmetic active ingredients.

Another object of the present invention is the aesthetic use, and therefore non-therapeutic use, of a hydrogel or composition according to the invention for preventing and/or treating the alteration of the viscoelastic or biomechanical properties of the skin, and in particular to regenerate, hydrate, firm or restore the radiance of the skin, in particular by mesotherapy; to fill volume defects of the skin, and in particular to fill wrinkles, fine lines or scars (in particular hollow scars); in order to reduce the appearance of wrinkles and fine lines; or, when said hydrogel or said composition comprises at least one cosmetic active ingredient, for the modified, delayed or prolonged release of cosmetic active ingredients, in particular as defined above.

For example, an object of present invention is the aesthetic use of a hydrogel or a composition according to the invention for attenuating the nasolabial folds and bitterness folds; for increasing the volume of the cheekbones, the chin or lips; for restoring the volumes of the face, in particular the cheeks, temples, the oval of the face, and around the eye; or to regenerate, hydrate, firm or restore the radiance of the skin, in particular by mesotherapy.

In particular, the hydrogel or the composition according to the invention is an anti-ageing hydrogel or composition. The hydrogel or the composition according to the invention is administered, more particularly, by injection.

The present invention also relates to a method for cosmetic treatment, preferably anti-ageing treatment, of keratin materials, in particular the skin, comprising at least one step of administering a hydrogel or a composition according to the invention on or through said keratin materials, more particularly by injection.

The administration can be an injection, in particular an intra-epidermal and/or intradermal and/or subcutaneous injection. The administration by intra-epidermal and/or intradermal and/or subcutaneous injection according to the invention aims to inject a hydrogel or a composition of the invention in an epidermal, dermoepidermal and/or dermal region. The hydrogel or the composition according to the invention can also be administered by a supra-periosteum injection.

The hydrogel or composition according to the invention can be injected using any one of the methods known to a person skilled in the art. In particular, a hydrogel or a composition according to the invention can be administered by means of an injection device suitable for an intraepidermal and/or intradermal and/or subcutaneous and/or supraperiosteal injection. The injection device can be chosen, in particular, from a syringe, a set of microsyringes, a laser or hydraulic device, an injection gun, a needle-free injection device, or a microneedle roller.

The injection device may have any commonly used injection means suitable for an intraepidermal and/or intradermal and/or subcutaneous and/or supra-periosteum injection. Preferably, such a means can be a hypodermic needle or a cannula.

A needle or cannula according to the invention can have a diameter varying from 18 to 34 G, preferably between 25 and 32 G, and a length varying from 4 to 70 mm, and preferably from 4 to 25 mm. The needle or cannula is advantageously disposable.

Advantageously, the needle or cannula is combined with a syringe or any other device enabling said injectable hydrogel or composition to be delivered through the needle or cannula.

According to an alternative embodiment, a catheter can be inserted between the needle/cannula and the syringe. In known manner, the syringe can be manually activated by the practitioner or even by a syringe support such as guns.

Preferably, the injection device can be chosen from a syringe or a set of micro-syringes. In an alternative embodiment, the injection device can be adapted to the mesotherapy technique.

Mesotherapy is a technique for treatment by intra-epidermal and/or intradermal and/or subcutaneous injection of a composition or hydrogel. The composition or the hydrogel is administered according to this technique by injection in the form of multiple small-sized droplets at the epidermis, the dermo-epidermal junction and/or the dermis in order, in particular, to produce a subcutaneous layer. The technique of mesotherapy is described, in particular, in the work entitled “Traite de mesotherapie” by Jacques LE COZ, published by Masson, 2004. Mesotherapy performed on the face is also called mesolift and also known by the term “mesoglow”.

The administration can be topical.

Preferably, it involves a topical application on the surface of the skin, more particularly on the epidermis, still more particularly on the facial epidermis.

The present invention also relates to an injection device as previously described comprising a hydrogel or a composition according to the invention.

The additional accessory biological effects of hydrogels according to the invention can be studied in vitro and/or in vivo and/or ex vivo; said in vivo tests can for example include administration tests, in small animals, of a composition according to the invention versus a comparative composition in order to follow the appearance of biological effects with, in particular, evaluation of the improvement in the quality of the skin in the animal, in particular the living human (e.g. its hydration and/or its elasticity) and, after sacrifice of the animal, histological sections in order to study any change in the protein expression at the site of administration (colouration).

These in vivo tests can generally include evaluating the quality of the skin in humans following the administration of a composition according to the invention vs. a comparative composition.

Said in vitro tests include tests on dermal cells (such as fibroblasts) for cytotoxicity, viability, protein expression (ELISA) in particular for the expression of hyaluronic acid, elastin, fibrillin, aquaporin and/or various types of collagen and genetic expression (e.g., genes coding for hyaluronic acid, elastin, fibrillin, aquaporin and/or various types of collagen).

The present invention is illustrated by the non-limiting examples below.

### EXAMPLES

### Materials and Methods

GPTMS: (3-Glycidyloxypropyl)trimethoxy-silane (Sigma 440167)
APTES: (3-aminopropyl)triethoxysilane (SIGMA)
NaHA: non-crosslinked sodium hyaluronate, 1.5 MDa and 4 MDa (HTL)
0.25 M NaOH
1 M HCl (Chem Lab)
Phosphate buffer PBS (Braun),
Lidocaine hydrochloride
Three-dimensional stirrer Turbula®
Paddle mill homogeniser
Sterile bag

### Measurement of Viscoelastic Properties

The viscoelastic properties of the prototypes obtained have been measured using a rheometer (DHR-2) having a stainless-steel cone (1°-40 mm) with cone-plane geometry and a peltier plane made of anodised aluminium (42 mm) (gap 24 μm).

For the measurements of elastic modulus G′, viscous modulus G″ and phase angle δ, a stress scan is carried out on the gel at 1 Hz and 25° C.

The elastic modulus G′, the viscous modulus G″ and the phase angle δ are reported for a stress of 5 Pa.

The stress at the intersection of G′ and G″, denoted T below, is determined at the intersection of the curves of the G′ and G″ moduli and is expressed in Pascal.

### Measurement of Cohesiveness

For the cohesiveness measurement (or mechanical strength, expressed in newtons), the gel is deposited on the Peltier plane with an initial gap of 2.60 mm. The gel is then compressed at constant speed of 100 μm/s to 70% of the initial gap, at 25° C. The cohesiveness of the gel is measured at the end of the compression stroke.

### Measurement of the Extrusion Force

The extrusion forces (in newtons) of prototypes packaged in syringes were measured using a test bench (Mecmesin 2.5-dV) equipped with a dynamometer (Mecmesin AFG 100N) at a constant speed of 12.5 mm/min, over a distance of 2.5 cm, through a 27 G ½″ needle and at ambient temperature. The extrusion force results correspond to the average of the average extrusion forces on at least 3 samples.

### Qualitative Study of the Stretchiness Property of a Product

1 mL of studied product is deposited on a smooth hard surface, e.g. a laboratory bench. Using his index finger, the operator exerts a pressure on the product so as to compress it against the surface then raises his index finger at a speed of order cm/sec, to a height of approximately 2 cm from the surface. A photograph is taken when the index finger is at a height of approximately 2 cm from the surface. If the gel is broken, the gel is not stretchy and has a low stretchiness property. If the gel is not broken, it has an acceptable stretchiness property.

Unless otherwise stated, the steps described below are carried out at ambient temperature (21° C.).

One month designates 31 days.

### Example 1: Study of the Influence of the Solid or Liquid State of the Reaction Medium on the Functionalisation and Sol-Gel Reaction

Prototype 1 (comparative) and prototype 2 according to the invention are each prepared in the following manner:
1—Dry 4 MDa sodium hyaluronate (NaHA) is dissolved at a concentration of 12% by weight in a 0.25 M sodium hydroxide solution, in a sterile bag,
2—The mixture is homogenised,
3—GPTMS is added in the sterile bag at a level of 0.35 moles of GPTMS per 1 mole of NaHA repetition unit,
4—The mixture is homogenised for 30 minutes,
5—The bag containing the mixture is:
   for prototype 1: maintained at 21° C., for 72 hours, the reaction medium is then in liquid form, the pH of the mixture is approximately 13,
   for prototype 2: placed in the freezer at −20° C., for 2 months, the reaction medium is in the solid state, the pH of the mixture is approximately 13, then the bag is thawed at ambient temperature by taking the bag out of the freezer and leaving it at ambient temperature until return to ambient temperature of the mixture in the bag,
6—A 1 N solution of HCl is added to the sterile bag until a pH of 7±0.5 was obtained,
7—The mixture is diluted with water for injectable preparation up to a concentration of 20 mg hyaluronic acid/g of gel,
8—The mixture is homogenised for 16 hours using a three-dimensional stirrer,
9—The mixture is dialysed,
10—The gel thus obtained is packaged in a syringe,
11—Finally, the gel is sterilised with the autoclave (F0>15).

The viscoelastic properties of each prototype are analysed. The results are presented in table 1 below:

TABLE 1
results.

| | | G′ (Pa) | | |
|---|---|---|---|---|
| | Duration and | after | | |
| | temperature | steril- | | |
| Prototype | of step 5 | isation | δ (°) | τ (Pa) |
| 1 | 72 hours, | 34 ± 6 | 40.4 ± 3.9 | 29 ± 17 |
| (comparative) | 21° C. | | | |
| 2 | 2 months, | 221 ± 21 | 19.1 ± 2.9 | 208 ± 25 |
| (invention) | −20° C. | | | |

When the reaction medium from the concomitant steps of functionalisation and sol-gel reaction is liquid, at ambient temperature (prototype 1—comparative), the prepared gel has lower viscoelastic properties (lower G′ and stress at the intersection of G′ and G″, higher phase angle δ) than when the medium is solid, frozen according to the invention, at a temperature of −20° C. (prototype 2 —in accordance with the invention).

Only the gel according to the invention (prototype 2) has acceptable properties for the intended therapeutic, cosmetic and aesthetic applications of the present invention (stress at the intersection of G′ and G″ greater than 50 Pa (208±25 Pa) and G′ greater than 20 Pa (221±21 Pa)). By contrast, the comparative gel (prototype 1) does not have acceptable properties (stress at the intersection of G′ and G″ less than 50 Pa (29±17 Pa)).

The state, liquid or solid, of the reaction medium during the concomitant steps of functionalisation and sol-gel reaction therefore has an influence on the viscoelastic properties of the prepared gels.

### Example 2: Influence of Temperature on the Preparation of Hydrogels According to the Invention

Prototypes 3 (according to the invention), 4 (comparative) and 5 (comparative) are each prepared in the following manner:
1—Dry 1.5 MDa sodium hyaluronate (NaHA) is dissolved at a concentration of 12% by weight in a 0.25 M sodium hydroxide solution, in a sterile bag,
2—GPTMS is added in the sterile bag at a level of 0.35 moles of GPTMS per 1 mole of NaHA repetition unit,
3—The mixture is homogenised,
4—The bag containing the mixture is:
   for prototype 3: placed in freezer at −20° C., for 1 month, the pH of the mixture is approximately 13, the reaction medium is then in the solid state, then the bag is thawed at ambient temperature by taking the bag out of the freezer and leaving it at ambient temperature until return to ambient temperature of the mixture in the bag,
   for prototype 4: maintained at 21° C., for 72 hours, the reaction medium is then in liquid form, the pH of the mixture is approximately 13,
   for prototype 5: maintained at 52° C., for 3 hours, the reaction medium is then in liquid form, the pH of the mixture is approximately 13,
5—A 1 N solution of HCl is added to the sterile bag until a pH of 7±0.5 was obtained,
6—The mixture is diluted with a phosphate-buffered saline to a concentration of 23 mg of hyaluronic acid/g of gel,
7—The mixture is homogenised for at least 16 hours and up to 24 hours using a three-dimensional stirrer,
8—The mixture is dialysed,
9—1% by weight of a phosphate-buffered saline solution at 30% by weight lidocaine hydrochloride and in order to counterbalance the acid pH of the lidocaine hydrochloride, and 0.4% by weight of a 0.25 M solution of NaOH are added to the dialysed mixture,
10—A fixed quantity of 4 MDa non-crosslinked NaHA is added as a lubricant,
11—The gel thus obtained is sieved,
12—The gel thus obtained is packaged in a syringe,
13—Finally, the gel is sterilised with the autoclave (F0>15).

The viscoelastic properties of each prototype were analysed. The results are presented in table 2 below:

TABLE 2
results.

| | Duration and | G′ (Pa) | | | | |
|---|---|---|---|---|---|---|
| | temperature of | after | | T | | Cohesive- |
| Prototype | step 4 | sterilisation | δ (°) | (Pa) | F(N) | ness (N) |
| 3 | 1 months, | 176 ± 29 | 23.6 ± 0.9 | 293 ± 30 | 21.4 ± 1.0 | 8.20 |
| (invention) | −20° C. | | | | | |
| 4 | 72 hours, 21° C. | 76 ± 5 | 27.0 ± 1.3 | 169 ± 23 | 18.1 ± 2.1 | 5.76 |
| (comparative) | | | | | | |
| 5 | 3 hours, 52° C. | 6 ± 2 | 59.1 ± 6.7 | N/A | 7.1 ± 0.2 | N/A |
| (comparative) | | | | | | |

Only prototypes 3 and 4 are gels (phase angle less than 45°), injectable (extrusion forces between 5 and 25 N) and have acceptable properties for the therapeutic, cosmetic and aesthetic applications targeted by the present invention (stress at the intersection of G′ and G″ greater than 50 Pa and G′ greater than 20 Pa).

Nevertheless, for identical concentrations of reactants (GPTMS, NaHA), the prototype according to the invention (prototype 3) has higher viscoelastic properties and cohesiveness than the prototypes prepared according to the comparative methods (prototype 4 and 5).

In a similar manner to example 1, when the reaction medium is frozen in accordance with the invention, at a temperature of −20° C. (prototype 3), the gel prepared has a higher G′ and higher stress at the intersection of G′ and G″ as well as a lower phase angle than a prototype prepared with a reaction medium that is liquid, at ambient temperature or at 52° C. (prototypes 4 and 5 —comparative).

The state, liquid or solid, of the reaction medium during the concomitant steps of functionalisation and sol-gel reaction therefore have an influence on the viscoelastic properties of the prepared gels.

### Example 3: Method According to the Invention for Preparing a Hydrogel Based on Hyaluronic Acid and GPTMS Comprising a Step of Crosslinking with a Crosslinking Agent

Prototypes 6 (comparative) and 7 to 9 (according to the invention) are each prepared in the following way:
1—Dry 1.5 MDa sodium hyaluronate (NaHA) is dissolved at a concentration of 12% by weight in a 0.25 M sodium hydroxide solution, in a sterile bag,
2—The GPTMS is added in the sterile bag at a level of 0.21 moles of GPTMS per 1 mole of NaHA repetition unit and 0.004 moles of BDDE per 1 mole of NaHA repetition unit,
3—The mixture is homogenised for 30 minutes,
4—The bag containing the mixture is:
   for prototype 6: maintained at 21° C., for 72 hours, the reaction medium is then in liquid form, the pH of the mixture is approximately 13,
   for prototype 7: placed in freezer at −20° C., for 21 days, the pH of the mixture is approximately 13, the reaction medium is then in the solid state, then the bag is thawed at ambient temperature by taking the bag out of the freezer and leaving it at ambient temperature until return to ambient temperature of the mixture in the bag,
   for prototype 8: placed in freezer at −20° C., for 30 days, the pH of the mixture is approximately 13, the reaction medium is then in the solid state, then the bag is thawed at ambient temperature by taking the bag out of the freezer and leaving it at ambient temperature until return to ambient temperature of the mixture in the bag,
   for prototype 9: placed in freezer at −20° C., for 2 months, the pH of the mixture is approximately 13, the reaction medium is then in the solid state, then the bag is thawed at ambient temperature by taking the bag out of the freezer and leaving it at ambient temperature until return to ambient temperature of the mixture in the bag.
5—A 1 N solution of HCl is added to the sterile bag until a pH of 7±0.5 was obtained,
6—The mixture is diluted with the phosphate-buffered saline to a concentration of 23 mg of HA/g of gel,
7—The mixture is homogenised for at least 16 hours and up to 24 hours using a three-dimensional stirrer,
8—The mixture is dialysed,
9—1% by weight of a phosphate-buffered saline solution at 30% by weight lidocaine hydrochloride and in order to counterbalance the acid pH of the lidocaine hydrochloride, 0.4% by weight of a 0.25 M solution of NaOH are added to the dialysed mixture,
10—A fixed quantity of 4 MDa non-crosslinked NaHA is added as a lubricant,
11—The gel thus obtained is sieved,
12—The gel thus obtained is packaged in a syringe,
13—Finally, the gel is sterilised with the autoclave (F0>15).

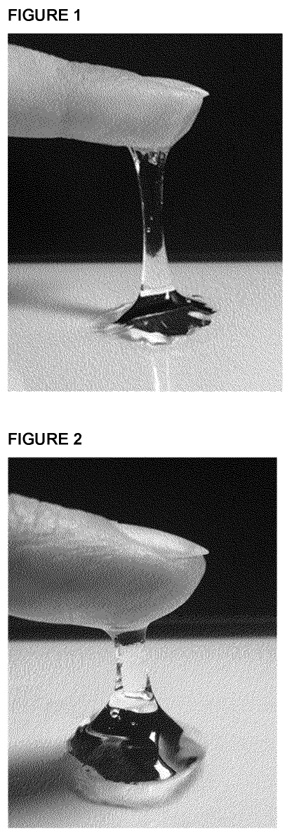

The viscoelastic properties of each prototype were analysed. The results are presented in table 3 below. In addition, the stretchiness property of each prototype is studied qualitatively. The results obtained for prototypes 6 to 9 are shown respectively in FIGS. 1 to 4.

FIG. 1 shows a photograph of the qualitative study of the stretchiness property of prototype 6.

FIG. 2 shows a photograph of the qualitative study of the stretchiness property of prototype 7.

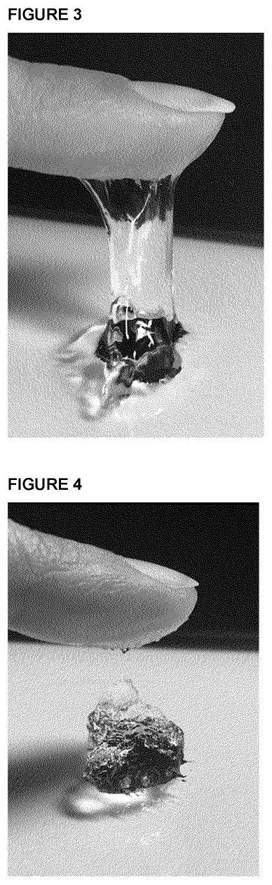

FIG. 3 shows a photograph of the qualitative study of the stretchiness property of prototype 8.

FIG. 4 shows a photograph of the qualitative study of the stretchiness property of prototype 9.

TABLE 3
results.

| | Duration | G′ (Pa) | | | |
|---|---|---|---|---|---|
| | and temper- | after | | | |
| | ature of | steril- | | | |
| Prototype | step 4 | isation | δ (°) | τ (Pa) | F(N) |
| 6 | 72 hours, | 38 ± 3 | 38.9 ± 1.1 | 51 ± 7 | 9.9 ± 0.5 |
| (compar- | 21° C. | | | | |
| ative) | | | | | |
| 7 | 21 days, | 119 ± 19 | 37.9 ± 2.2 | 143 ± 41 | 9.1 ± 0.3 |
| (inven- | −20° C. | | | | |
| tion) | | | | | |
| 8 | 30 days, | 129 ± 17 | 27.9 ± 1.6 | 216 ± 25 | 18.1 ± 0.5 |
| (inven- | −20° C. | | | | |
| tion) | | | | | |
| 9 | 2 months, | 326 ± 40 | 15.5 ± 1.6 | 395 ± 16 | 16.7 ± 1.3 |
| (inven- | −20° C. | | | | |
| tion) | | | | | |

All the prototypes 6 and 9 are gels (phase angles less than 45°), injectable (extrusion forces between 5 and 25 N) and have acceptable properties for the therapeutic, cosmetic and aesthetic applications targeted by the present invention (stresses at the intersection of G′ and G″ greater than 50 Pa and G′ greater than 20 Pa).

Nevertheless, the method according to the invention leads to the preparation of very stretchy gels (prototypes 7 to 9), which is not the case for the comparative method. This stretchiness/non-stretchiness property is visible in the respective photographs of prototypes 6 to 9 in FIGS. 1 to 4.

Prototypes 7, 8 and 9 according to the invention have higher elastic properties (higher G′ and lower phase angle) than the comparative prototype 6.

The elastic properties of prototypes 7 to 9 progressively increase, proportionally with the duration of the concomitant steps of functionalisation, sol-gel reaction and crosslinking.

The state, liquid or solid, of the reaction medium during the concomitant steps of functionalisation and sol-gel reaction therefore have an influence on the viscoelastic properties of the prepared gels.

### Example 4: Methods for Preparing Gels Based on Hyaluronic Acid and GPTMS Using Different Functionalisation Ratios with GPTMS

Prototypes 10 to 12 according to the invention are each prepared as follows:
1—Dry 1.5 MDa sodium hyaluronate (NaHA) is dissolved at a concentration of 12% by weight in a 0.25 M sodium hydroxide solution, in a sterile bag,
2—The GPTMS is added in the sterile bag respectively at a level of:
   for prototype 10: 0.1 moles of GPTMS per 1 mole of NaHA repetition unit,
   for prototype 11: 0.21 moles of GPTMS per 1 mole of NaHA repetition unit,
   for prototype 12: 0.35 moles of GPTMS per 1 mole of NaHA repetition unit,
3—The mixture is homogenised,
4—The bag containing the mixture is placed in the freezer at −20° C. for 1 month, except for that of prototype 10 which is left for 3 months: the pH of the mixture is approximately 13, the reaction medium is then in the solid state, then the bag is thawed at ambient temperature by taking the bag out of the freezer and leaving it at ambient temperature until return to ambient temperature of the mixture in the bag,
5—A 1 N solution of HCl was added to the sterile bag until a pH of 7±0.5 was obtained,
6—The mixture is diluted with the phosphate-buffered saline to a concentration of 23 mg of HA/g of gel,
7—The mixture is homogenised for at least 16 hours and up to 24 hours using a three-dimensional stirrer,
8—The mixture is dialysed,
9—1% by weight of a phosphate-buffered saline solution at 30% by weight lidocaine hydrochloride and in order to counterbalance the acid pH of the lidocaine hydrochloride, and 0.4% by weight of a 0.25 M solution of NaOH are added to the dialysed mixture;
10—A fixed quantity of 4 MDa non-crosslinked NaHA is added as a lubricant,
11—The gel thus obtained is sieved,
12—The gel obtained is packaged in a syringe,
13—Finally, the gel is sterilised with the autoclave (F0>15).

The viscoelastic properties of each prototype were analysed. The results are presented in table 4 below:

TABLE 4
results.

| | GPTMS | | | | | |
|---|---|---|---|---|---|---|
| | functionalisation | Duration | G′ (Pa) after | | T | |
| Prototype | ratio (% molar) | of step 4 | sterilisation | δ (°) | (Pa) | F(N) |
| 10 | 10 | 3 months | 126 ± 20 | 33.9 ± 2, | 192 ± 40 | 16.7 ± 0.6 |
| 11 | 21 | 1 month | 73 ± 7 | 42.8 ± 1.3 | 59 ± 22 | 9.6 ± 0.2 |
| 12 | 35 | 1 month | 176 ± 29 | 23.6 ± 0.9 | 293 ± 30 | 21.4 ± 1.0 |

All the prototypes 10 and 12 are gels (phase angles less than 45°), injectable (extrusion forces between 5 and 25 N) and have acceptable properties for the therapeutic, cosmetic and aesthetic applications targeted by the present invention (stresses at the intersection of G′ and G″ greater than 50 Pa and G′ greater than 20 Pa).

In view of the results above, the elastic properties of the prototypes increase with the quantity of GPTMS used for a given freezing time (G′ prototype 11, 21% molar of GPTMS=73±7 Pa<G′ prototype 12, 35% molar of GPTMS=176±29 Pa).

### Example 5: Method for Preparing a Hydrogel Based on Hyaluronic Acid and GPTMS in which Only the Advanced Condensation Step is Carried Out at −20° C.

Prototypes 13 and 14 according to the invention are each prepared as follows:
1—Dry 1.5 MDa sodium hyaluronate (NaHA) is dissolved at a concentration of 12% by weight in a 0.25 M sodium hydroxide solution, in a sterile bag,
2—GPTMS is added in the sterile bag respectively at a level of 0.35 moles of GPTMS per 1 mole of NaHA repetition unit,
3—The mixture is homogenised,
4—The bag containing the mixture is placed at 21° C. for 3 days, the pH of the mixture is approximately 13,
5—Then
   for prototype 13: the bag containing the mixture is placed at −20° C., for 1 month, the pH of the mixture is approximately 13, the reaction medium is then in solid form; the mixture is then thawed at ambient temperature by taking the bag out of the freezer and leaving it at ambient temperature until return to ambient temperature of the mixture in the bag,
   for prototype 14: step 6 follows step 4,
6—A 1 N solution of HCl is then added to the sterile bag until a pH of 7±0.5 is obtained,
7—The mixture is diluted with the phosphate-buffered saline to a concentration of 23 mg of HA/g of gel,
8—The mixture is homogenised for at least 16 hours and up to 24 hours using a three-dimensional stirrer,
9—The mixture is dialysed,
10—1% by weight of a phosphate-buffered saline solution at 30% by weight lidocaine hydrochloride and in order to counterbalance the acid pH of the lidocaine hydrochloride, 0.4% by weight of a 0.25 M solution of NaOH are added to the dialysed mixture,
11—A fixed quantity of 4 MDa non-crosslinked NaHA is added as a lubricant,
12—The gel thus obtained is sieved then packaged in a syringe,
13—Finally, the gel is sterilised with the autoclave (F0>15).

The viscoelastic properties of each prototype were analysed. The results are presented in table 5 below:

TABLE 5
results.

| | Duration and | | | | |
|---|---|---|---|---|---|
| | temperature | | | | |
| Prototype | of step 5 | G′ (Pa) | δ (°) | τ (Pa) | F(N) |
| 13 | −20° C., | 182 ± 16 | 15.7 ± 0.6 | 426 ± 25 | 19.8 ± 0.8 |
| | 1 month | | | | |
| 14 | N/A | 76 ± 5 | 27.0 ± 1.3 | 169 ± 23 | 18.1 ± 2.1 |

The functionalisation and freezing steps have been separated in order to visualise their influence on the properties of the gel. It can thus be observed that the freezing step can improve the sol-gel reaction and thus obtain a gel with greater elastic properties.

### Example 6: Influence of the Molecular Weight of the Polysaccharide

Prototypes 15 to 18 according to the invention are each prepared as follows:
1—Dry 1.5 MDa sodium hyaluronate (NaHA) for prototypes 15 and 16, or dry 4 MDa NaHA for prototypes 17 and 18 is dissolved at a concentration of 12% by weight in a 0.25 M sodium hydroxide solution, in a sterile bag,
2—GPTMS is added in the sterile bag at a level of 0.21 moles of GPTMS per 1 mole of NaHA repetition unit,
3—The mixture is homogenised,
4—The bag containing the mixture is placed in the freezer at −20° C. for 1 month (for prototypes 15 and 17) or 2 months (for prototypes 16 and 18), the pH of the mixture is approximately 13, the reaction medium is then in the solid state,
5—The mixture is thawed at ambient temperature by taking the bag out of the freezer and by leaving it at ambient temperature until return to ambient temperature of the mixture in the bag,
6—A 1 N solution of HCl was added to the sterile bag until a pH of 7±0.5 was obtained,
7—The mixture is diluted with the phosphate-buffered saline to a concentration of 23 mg of HA/g of gel,
8—The mixture is homogenised for at least 16 hours and up to 24 hours using a three-dimensional stirrer,
9—The mixture is dialysed,
10—1% by weight of a phosphate-buffered saline solution at 30% by weight lidocaine hydrochloride and in order to counterbalance the acid pH of the lidocaine hydrochloride, 0.4% by weight of a 0.25 M solution of NaOH are added to the dialysed mixture,
11—A fixed quantity of 4 MDa non-crosslinked NaHA is added as a lubricant,
12—The gel thus obtained is sieved then packaged in a syringe,
13—Finally, the gel is sterilised with the autoclave (F0>15).

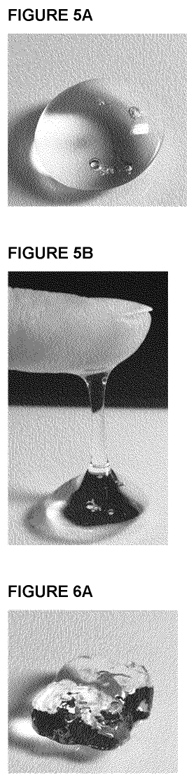

The viscoelastic properties of each prototype were analysed. The results are presented in table 6 below and the study of the stretchiness property is visible in the respective photographs of prototypes 15 to 18 in FIGS. 5A, 5B, 6A, 6B, 7A, 7B, 8A and 8B.

FIG. 5A shows the photograph of 1 mL of prototype 15 deposited on a smooth hard surface before the qualitative study of its stretchiness property.

FIG. 5B shows a photograph of the qualitative study of the stretchiness property of prototype 15.

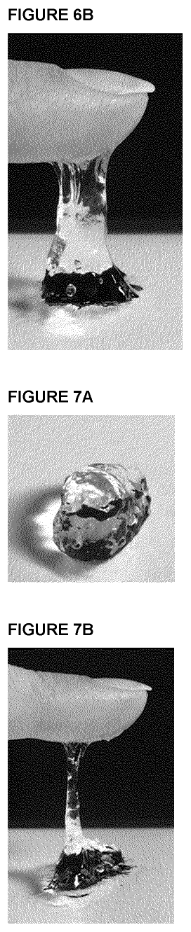

FIG. 6A shows the photograph of 1 mL of prototype 16 deposited on a smooth hard surface before the qualitative study of its stretchiness property.

FIG. 6B shows a photograph of the qualitative study of the stretchiness property of prototype 16.

FIG. 7A shows the photograph of 1 mL of prototype 17 deposited on a smooth hard surface before the qualitative study of its stretchiness property.

FIG. 7B shows a photograph of the qualitative study of the stretchiness property of prototype 17.

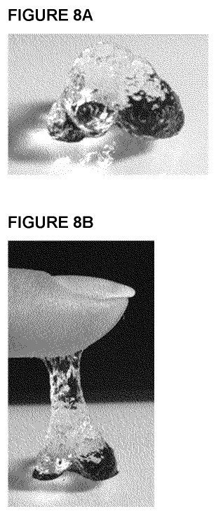

FIG. 8A shows the photograph of 1 mL of prototype 18 deposited on a smooth hard surface before the qualitative study of its stretchiness property.

FIG. 8B shows a photograph of the qualitative study of the stretchiness property of prototype 18.

TABLE 6
results.

| | Mw of | GPTMS | | | | |
|---|---|---|---|---|---|---|
| | the HA | functionalisation | Duration | G′ (Pa) after | | T |
| Prototype | (MDa) | ratio (% molar) | of step 4 | sterilisation | δ (°) | (Pa) |
| 15 | 1.5 | 21 | 1 months | 73 ± 7 | 42.8 ± 1.3 | 59 ± 22 |
| 16 | 1.5 | 21 | 2 months | 138 ± 18 | 26.3 ± 1.7 | 215 ± 22 |
| 17 | 4 | 21 | 1 months | 251 ± 37 | 23.9 ± 1.3 | 322 ± 40 |
| 18 | 4 | 21 | 2 months | 347 ± 20 | 21.2 ± 0.9 | 354 ± 36 |

All the prototypes 15 to 18 are gels (phase angles less than 45°).

All the prototypes 15 to 18 have acceptable properties for the therapeutic, cosmetic and aesthetic applications targeted by the present invention (stress at the intersection of G′ and G″ greater than 50 Pa and G′ greater than 20 Pa).

The results above therefore show that the method according to the invention functions using NaHA of different molecular weights (1.5 MDa and 4 MDa).

It is also clear from the above results that by increasing the duration of the concomitant steps of functionalisation and sol-gel reaction according to the invention at −20° C. from 1 to 2 months, the gel properties are reinforced and this is the case whatever the molecular weight of the NaHA initially used.

By comparing prototypes 15 and 16 and 17 and 18 with one another, it is clear that the gels produced with 4 MDa NaHA have both a larger G′, a larger stress at the intersection of G′ and G″ (T) and a smaller phase angle δ, i.e. a larger elastic component, than the gels produced with the 1.5 MDa NaHA. It is therefore possible to obtain gels with greater mechanical strength by using a higher molecular weight NaHA compared with a gel prepared according to the same method but using a lower molecular weight NaHA.

Consequently, only by using a 4 MDa NaHA in place of a 1.5 MDa NaHA is it possible to reduce the quantity of functionalisation agent necessary (in this case GPTMS) and/or to reduce the duration of the concomitant steps of functionalisation and sol-gel reaction in order to obtain a gel with mechanical properties identical to the final.

### Example 7: Methods for Preparing Gels Based on Hyaluronic Acid and APTES

Prototypes 19 and 20 according to the invention are each prepared as follows:
1—Dry 1.5 MDa sodium hyaluronate (NaHA) is dissolved at a concentration of 6.7% by weight in a 0.25 M sodium hydroxide solution, in a pot,
2—The 1-ethyl-3-(3-dimethylaminopropyl)carbodiimide (EDC) and N-hydroxysuccinimide (NHS) are dissolved in 3.5 mL of water for injectable preparation in order to obtain, for each of these two reagents, a molar ratio in moles of reactants per mole of hyaluronic acid disaccharide unit of 0.5. The solution obtained is added to the hyaluronic acid solution. The mixture is stirred manually with a spatula for 5 minutes then left to react for 30 minutes. The pH is then controlled and, if necessary, adjusted to attain a pH equal to 6 with 1 M HCl 1M or 0.25 M NaOH,
3—The APTES is added dropwise to the mixture of hyaluronic acid, EDC and NHS, in order to obtain a molar ratio in moles of APTES per mole of hyaluronic acid disaccharide unit of 0.2. The pH is controlled and, if necessary adjusted to reach a pH between 4.5 and 6.5 with 1 M HCl 1M or 0.25 M NaOH,
4—The product is then placed in the oven for 15 hours at 21° C.,
5—At the end of these 15 hours, the product is, if necessary, brought to a pH between 6.8 and 7.8,
6—Then, prototype 20 is placed for 48 hours at −20° C., while prototype 19 is directly treated according to the following steps,
7—The product is diluted with the phosphate-buffered saline to a concentration of 23 mg of HA/g of gel,
8—The mixture is homogenised for at least 16 hours and up to 24 hours using a three-dimensional stirrer,
9—The mixture is dialysed,
10—A fixed quantity of 4 MDa non-crosslinked NaHA is added as a lubricant,
11—The gel thus obtained is sieved,
12—the gel obtained is packaged in a syringe,
13—Finally, the gel is sterilised with the autoclave (F0>15).

The viscoelastic properties of each prototype were analysed. The results are presented in table 7 below:

TABLE 7
results

| | | G′ (Pa) | | |
|---|---|---|---|---|
| | | after | | |
| | Duration | steril- | | |
| Prototype | of step 5 | isation | δ (°) | τ (Pa) |
| 19 | 0 | 90 ± 7 | 47.0 ± 1.0 | N/A |
| 20 | 48 hours | 126 ± 5 | 35.1 ± 0.6 | 153 ± 9 |

While a condensation reaction by a single passage at a pH of 6.8 to 7.8 is not sufficient to prepare a gel with properties acceptable for the applications targeted by the present invention (prototype 19 has a viscous predominance with a 5 greater than 45°, a modulus G″ greater than modulus G′ including for low stresses and therefore no intersection of the curves of G′ and G″), carrying out of this condensation reaction in the frozen state (−20° C., atmospheric pressure) and at a pH of 6.8 to 7.8, enables it (prototype 20 with stress at the intersection of G′ and G″ greater than 50 Pa and G′ greater than 20 Pa).

1. A method for preparing a hydrogel comprising the following steps:
a) providing at least one polysaccharide;
b) providing at least one molecule of formula Chem. I: 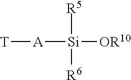
or a salt thereof,
wherein:
T represents an isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy or carbodiimide group, or an acid anhydride residue;
A represents a chemical bond or a spacer group;
R⁵ and R⁶, identical or different, represent a hydrogen atom; a halogen atom; an —OR⁴ group with R⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl;
R¹⁰ represents a hydrogen atom, an aryl group or an aliphatic hydrocarbon group having 1 to 6 carbon atoms;
c) functionalisation of the polysaccharide with at least one molecule of formula Chem. I;
d) crosslinking by sol-gel reaction of the functionalised polysaccharide in order to give a hydrogel;
wherein step d) comprises a crosslinking by sol-gel reaction carried out in a reaction medium at a pH greater than or equal to 9 and less than 14, at a pressure P less than or equal to atmospheric pressure and at a temperature T greater than the temperature of the eutectic point of the reaction medium as measured at pressure P and less than the temperature of the freezing point of the reaction medium as measured at pressure P, for a duration t ranging from 2 weeks to 17 weeks, or
wherein step d) comprises a crosslinking by sol-gel reaction carried out in a reaction medium at a pH greater than or equal to 6.8 and less than or equal to 7.8, at a pressure P less than or equal to the atmospheric pressure and at a temperature T greater than the temperature of the eutectic point of the reaction medium as measured at pressure P and less than the temperature of the freezing point of the reaction medium as measured at pressure P, for a duration t between 1 hour and 48 hours.

2. The method according to claim 1, wherein T is between −55° C. and −5° C.

3. The method according to claim 1, wherein the polysaccharide is chosen from pectin and pectic substances; chitosan; cellulose and its derivatives; agarose; glycosaminoglycans; and the mixtures thereof.

4. The method according to claim 1 wherein the polysaccharide is a glycosaminoglycan.

5. The method according to claim 1 wherein in the molecule of formula Chem. I A is a divalent aliphatic hydrocarbon chain having 1 to 12 carbon atoms, wherein one or more divalent units chosen from the arylenes, —O—, —S—, —S(O)—, —C(═O)—, —SO₂— and —N(R⁹)— are optionally intercalated between two carbon atoms of said chain, with R⁹ representing a hydrogen atom, an aliphatic hydrocarbon group having 1 to 6 carbon atoms, or an aryl-(C1-C6)alkyl, said chain being non-substituted or substituted by one or more monovalent groups chosen from a halogen atom, a hydroxide, an aryl-(C1-C6)alkyl.

6. The method according to claim 1 wherein in the molecule of formula Chem. I:
A is a divalent chain —(C1-C6)alkylene-O—(C1-C6)alkylene-;
R⁵ and R⁶, identical or different, are each an —OR⁴ group with R⁴ representing a (C1-C6)alkyl group; or a (C1-C6)alkyl group, and
R¹⁰ is a (C1-C6)alkyl group.

7. The method according to claim 1, wherein in step c), said polysaccharide is functionalised in the presence of 0.01 to 0.5, moles of molecule of formula Chem. I or a salt thereof, per 1 mole of polysaccharide repetition unit.

8. The method according to claim 1, wherein steps c) and d) are concomitant or partially concomitant.

9. The method according to claim 1, further comprising a step e) of crosslinking of the polysaccharide in the presence of at least one crosslinking agent or a salt thereof, said crosslinking agent comprising at least two functional groups Z, identical or different, chosen from the isocyanate, amino, epoxide, carboxyl, N-succinimidyloxycarbonyl, N-sulfosuccinimidyloxycarbonyl, halogenocarbonyl, isothiocyanate, vinyl, formyl, hydroxyl, sulfhydryl, hydrazino, acylhydrazino, aminoxy, carbodiimide groups, and an anhydrous acid residue.

10. The method according to claim 1, further comprising a step f) of adding a molecule of formula Chem. III: R⁷O—[R¹²R¹³SiO]ₚ—R⁸
or a salt thereof
wherein:
p is an integer from 1 to 20;
R¹² and R¹³, identical or different, represent a hydrogen atom; a halogen atom; an —OR¹⁴ group with R¹⁴ representing a hydrogen atom, an aryl group or an aliphatic hydrocarbon group having 1 to 6 carbon atoms; an aryl; or an aliphatic hydrocarbon group having 1 to 6 carbon atoms optionally substituted by one or more groups chosen from a halogen atom, an aryl and a hydroxyl; and
R⁷ and R⁸, identical or different, represent a hydrogen atom, an aryl group or an aliphatic hydrocarbon group having 1 to 6 carbon atoms.

11. The method according to claim 1, further comprising a step j) of sterilising the hydrogel.

12. The method according to claim 1, further comprising a step g) of adding at least one additional component chosen from lubricants; cosmetic active ingredients such as antioxidants, co-enzymes, amino acids, vitamins, minerals, and nucleic acids; and the mixtures thereof.

13. The method according to claim 1, comprising the addition of at least one therapeutic active ingredient.

14. A hydrogel that can be obtained by a method according to claim 1.

15. A cosmetic or pharmaceutical composition comprising a hydrogel according to claim 14 and a physiologically acceptable excipient.

16. A method for filling and/or replacing tissues comprising administering to a subject the hydrogel according to claim 14 or a composition comprising the hydrogel of claim 14 and a physiologically acceptable excipient.

17. An aesthetic method for preventing and/or treating the change in the viscoelastic or biomechanical properties of the skin; for filling volume defects of the skin; for attenuating the nasolabial folds and bitterness folds; for increasing the volume of the cheekbones, the chin or lips; for restoring the volumes of the face, and around the eye; for reducing the appearance of wrinkles and fine lines; or to regenerate, hydrate, firm or restore the radiance of the skin comprising administering to a subject the hydrogel according to claim 14 or a composition comprising the hydrogel according to claim 14 and a physiologically acceptable excipient.

18. An aesthetic method for the modified delayed or prolonged release of at least one cosmetic active ingredient comprising administering to a subject a hydrogel according to claim 14 or comprising the hydrogel according to claim 14 and a physiologically acceptable excipient, the hydrogel or the composition comprising the at least one cosmetic active ingredient.

19. The method according to claim 4, wherein the polysaccharide is hyaluronic acid or a salt thereof.

20. The method according to claim 6, wherein in the molecule of formula Chem. I, T represents an amino or epoxide group.
